(19) United States
(12) Patent Application Publication
(10) Pub. No.: US 2009/0306037 A1
(43) Pub. Date: Dec. 10, 2009

(54) Combination of a Dipeptidyl Peptidase-IV Inhibitor and a Cannabinoid CB1 Receptor Antagonist for the Treatment of Diabetes and Obesity

(75) Inventors: John M. Amatruda, New York, NY (US); Tung M. Fong, Somerset, NJ (US); David E. Moller, Bedminster, NJ (US); Nancy A. Thornberry, Westfield, NJ (US)

Correspondence Address: MERCK AND CO., INC, P O BOX 2000, RAHWAY, NJ, 07065-0907, US

(73) Assignee: MERCK & CO., INC., Rahway, NJ (US)

(21) Appl. No.: 11/919,308

(22) PCT Filed: Apr. 28, 2006
(86) PCT No.: PCT/US2006/016754

§ 371 Date: Oct. 26, 2007

Related U.S. Application Data
(60) Provisional application No. 60/676,783, filed on May 2, 2005.

(51) Int. Cl. A61K 31/4985; A61P 3/06; A61P 3/10

(52) U.S. Cl. 514/210.01; 514/249

(57) ABSTRACT

The present invention relates to pharmaceutical compositions comprising a combination of a particular dipeptidyl peptidase-IV (DPP-IV) inhibitor and a particular cannabinoid CB?1#191 receptor antagonist/inverse agonist, kits containing such combinations and methods of using such compositions for the treatment of diabetes, diabetes associated with obesity, diabetes-related disorders, obesity, and obesity-related disorders.

### FIELD OF THE INVENTION

The present invention relates to pharmaceutical compositions comprising a combination of a particular dipeptidyl peptidase-IV (DPP-IV) inhibitor and a particular cannabinoid CB₁ receptor antagonist/inverse agonist, kits containing such combinations and methods of using such compositions for the treatment of diabetes, diabetes associated with obesity, diabetes-related disorders, obesity, and obesity-related disorders.

### BACKGROUND OF THE INVENTION

Diabetes is caused by multiple factors and is most simply characterized by elevated levels of plasma glucose (hyperglycemia) in the fasting or post glucose-challenge state. There are two generally recognized forms of diabetes: Type 1 diabetes, or insulin-dependent diabetes mellitus (IDDM), in which patients produce little or no insulin, the hormone which regulates glucose utilization, and Type 2 diabetes, or noninsulin-dependent diabetes mellitus (NIDDM), wherein patients produce some insulin and even exhibit hyperinsulinemia (plasma insulin levels that are the same or even elevated in comparison with non-diabetic subjects), while at the same time demonstrating hyperglycemia. Type 1 diabetes is typically treated with exogenous insulin administered via injection. However, Type 2 diabetics often develop “insulin resistance”, such that the effect of insulin in stimulating glucose and lipid metabolism in the main insulin-sensitive tissues, namely, muscle, liver and adipose tissues, is diminished. Patients who are insulin resistant but not diabetic may have elevated insulin levels that compensate for their insulin resistance, so that serum glucose levels are not elevated. In patients with Type 2 diabetes, the plasma insulin levels, even when they are elevated, are insufficient to overcome the pronounced insulin resistance, resulting in hyperglycemia.

Insulin resistance is primarily due to a post receptor signaling defect that is not yet completely understood. Resistance to insulin results in insufficient activation of glucose uptake, diminished oxidation of glucose and storage of glycogen in muscle, inadequate insulin repression of lipolysis in adipose tissue and inadequate suppression of glucose production by the liver.

The persistent or uncontrolled hyperglycemia that occurs in diabetics is associated with increased morbidity and premature mortality. Type 2 diabetics are at increased risk of developing cardiovascular complications, e.g., atherosclerosis, coronary heart disease, stroke, peripheral vascular disease, hypertension, nephropathy, neuropathy, and retinopathy.

Many patients who have insulin resistance but have not yet developed Type 2 diabetes are also at a risk of developing Metabolic Syndrome, also referred to as syndrome X, insulin resistance syndrome, or pluriMetabolic Syndrome. The period of 5 to 10 years preceding the development of impaired glucose tolerance is associated with a number of hormonal imbalances, which give rise to an enlargement of visceral fat mass, hypertension, insulin resistance, and hyperlipidemia (Bjornstop, P., Current Topics in Diabetes Research, eds. Belfore, F., Bergman, R. N., and Molinath, G. M., Front Diabetes, Basel, Karger, 12: 182-192 (1993)). Similarly, Metabolic Syndrome is characterized by insulin resistance, along with abdominal obesity, hyperinsulinemia, high blood pressure, low HDL and high VLDL. Although the causal relationship between the various components of Metabolic Syndrome remains to be confirmed, insulin resistance or abdominal obesity appears to play an important role (Requen, G. M., et al., N. Eng. J. Med., 334: 374-381 (1996); Despres, J-P., et al., N. Engl. J. Med., 334: 952-957 (1996); Wajchenberg, B. L., et al., Diabetes/Metabolism Rev., 10: 19-29 (1994)). Metabolic Syndrome patients, whether or not they develop overt diabetes mellitus, are at increased risk of developing the cardiovascular complications listed above. Diabetes can be treated with a variety of therapeutic agents, including insulin sensitizers, such as PPARγ agonists, such as glitazones and glitazars; biguanides; protein tyrosine phosphatase-1B inhibitors; dipeptidyl peptidase-IV inhibitors; insulin; insulin mimetics; sulfonylureas; meglitinides; α-glucosidase inhibitors; and α-amylase inhibitors.

Increasing the plasma level of insulin by administration of sulfonylureas (e.g. tolbutamide and glipizide) or meglitinides, which stimulate the pancreatic β-cells to secrete more insulin, and/or by injection of insulin when sulfonylureas or meglitinides become ineffective, can result in insulin concentrations high enough to stimulate insulin-resistant tissues. However, dangerously low levels of plasma glucose can result, and increasing insulin resistance due to the even higher plasma insulin levels can occur. The biguanides have an unknown mechanism of action but decrease hepatic glucose output and increase insulin sensitivity resulting in some correction of hyperglycemia. Metformin monotherapy is often used for treating Type 2 diabetic patients who are also obese and/or dyslipidernic. Lack of appropriate response to metformin is often followed by treatment with sulfonylureas, thiazolidinediones, insulin, or α-glucosidase inhibitors. However, the two biguanides, phenformin and metformin, can also induce lactic acidosis and nausea/diarrhea, respectively. α-Glucosidase inhibitors, such as acarbose, work by delaying absorption of glucose in the intestine. α-Amylase inhibitors inhibit the enzymatic degradation of starch or glycogen into maltose, which also reduces the amounts of bioavailable sugars.

The PPAR-γ agonists, including glitazones, also known as thiazolidinediones (i.e. 5-benzylthiazolidine-2,4-diones) and non-thiazolidinediones, i.e. glitizars, represent another class of compounds with potential for ameliorating many symptoms of Type 2 diabetes. These agents substantially increase insulin sensitivity in muscle, liver and adipose tissue in several animal models of Type 2 diabetes resulting in partial or complete correction of the elevated plasma levels of glucose without occurrence of hypoglycemia. The glitazones that are currently marketed are agonists of the peroxisome proliferator activated receptor (PPAR) gamma subtype. PPAR-gamma agonism is generally believed to be responsible for the improved insulin sensitization that is observed with the glitazones. Newer PPAR agonists that are being developed for treatment of Type 2 diabetes and/or dyslipidemia are agonists of one or more of the PPAR alpha, gamma and delta subtypes.

However, treatment of diabetes with PPARγ agonists has been associated with fluid retention. Recent labeling revisions for Avandia® (rosiglitazone maleate), a PPARγ agonist, indicate that patients may experience fluid accumulation and volume-related events such as edema and congestive heart failure.

Treatment of Type 2 diabetes also typically includes physical exercise, weight control, and dieting. While physical exercise and reductions in dietary intake of calories will dramatically improve the diabetic condition, compliance with this treatment is very poor because of well-entrenched sedentary lifestyles and excess food consumption, especially of foods containing high amounts of saturated fat. However, weight reduction and increased exercise are difficult for most people with diabetes.

Abnormal glucose homeostasis is also associated both directly and indirectly with obesity, hypertension and alterations in lipid, lipoprotein and apolipoprotein metabolism. Obesity increases the likelihood of insulin resistance, and increases the likelihood that the resulting insulin resistance will increase with increasing body weight. Therefore, therapeutic control of glucose homeostasis, lipid metabolism, obesity and hypertension are critically important in the prevention of and clinical management and treatment of diabetes mellitus.

Obesity, which can be defined as a BMI>30 kg/m2 for Caucasians or >25 kg/m2 for Asians, is a major health concern in Western societies. It is estimated that about 97 million adults in the United States are overweight or obese. Obesity is the result of a positive energy balance, as a consequence of increased ratio of caloric intake to energy expenditure. The molecular factors regulating food intake and body weight balance are incompletely understood [B. Staels et al., J. Biol. Chem., 270: 15958 (1995); F. Lonnquist et al., Nature Medicine, 1: 950 (1995)]. Although the genetic and/or environmental factors leading to obesity are poorly understood, several genetic factors have been identified.

Epidemiological studies have shown that increasing degrees of overweight and obesity are important predictors of decreased life expectancy. Obesity causes or exacerbates many health problems, both independently and in association with other diseases. The medical problems associated with obesity, which can be serious and life-threatening, include Type 2 diabetes mellitus; hypertension; elevated plasma insulin concentrations; insulin resistance; dyslipidemias; hyperlipidemia; endometrial, breast, prostate, kidney and colon cancer; osteoarthritis; respiratory complications, such as obstructive sleep apnea; gallstones; atherosclerosis; heart disease; abnormal heart rhythms; and heart arrythmias (Kopelman, P. G., Nature, 404: 635-643 (2000)). Obesity is also associated with Metabolic Syndrome, cardiac hypertrophy, in particular left ventricular hypertrophy, premature death, and with a significant increase in mortality and morbidity from stroke, myocardial infarction, congestive heart failure, coronary heart disease, and sudden death.

Abdominal obesity has been linked with a much higher risk of coronary artery disease, and with three of its major risk factors: high blood pressure, diabetes that starts in adulthood, and high levels of fats (lipids) in the blood. Losing weight dramatically reduces these risks. Abdominal obesity is further closely associated with glucose intolerance, hyperinsulinemia, hypertriglyceridemia, and other disorders associated with Metabolic Syndrome, such as raised high blood pressure, decreased levels of high density lipoproteins (HDL) and increased levels of very low density lipoproteins (VLDL) (Montague et al., Diabetes, 49: 883-888 (2000)).

Obesity and obesity-related disorders, such as diabetes, are often treated by encouraging patients to lose weight by reducing their food intake or by increasing their exercise level, thereby increasing their energy output. A sustained weight loss of 5% to 10% of body weight has been shown to improve the co-morbidities associated with obesity, such as diabetes, and can lead to improvement of obesity-related disorders, such as left ventricular hypertrophy, osteoarthritis, and pulmonary and cardiac dysfunction.

Weight loss drugs used for the treatment of obesity include orlistat [Davidson, M. H. et al., JAMA. 281: 235-42 (1999)], dexfenfluramine [Guy Grand, B. et al., Lancet. 2: 1142-5 (1989)], sibutramine [Bray, G. A. et al., Obes. Res., 7: 189-98 (1999)], and phentermine [Douglas, A. et al., Int. J. Obes., 7: 591-5 (1983)]. However, the side effects of these drugs and anti-obesity agents may limit their use. Dexfenfluramine was withdrawn from the market because of suspected heart valvulopathy; orlistat is limited by gastrointestinal side effects; and the use of sibutramine is limited by its cardiovascular side effects which have led to reports of deaths and its withdrawal from the market in Italy.

There is a continuing need for new methods of treating diabetes, diabetes associated with obesity, and diabetes-related disorders. There is also a need for new methods of treating and preventing obesity and obesity related disorders, such as Metabolic Syndrome. There is currently no approved treatment for Metabolic Syndrome.

The present invention addresses this problem by providing a combination therapy comprising of a particular dipeptidyl peptidase-IV (DPP-IV) inhibitor and a particular cannabinoid CB₁ antagonist/inverse agonist for the treatment of diabetes, diabetes associated with obesity, diabetes-related disorders, obesity, and obesity-related disorders. The combination of these agents, at their respective clinical doses, is expected to be more effective than treatment with either agent alone. Treatment with such a combination at sub-clinical doses is expected to produce clinical efficacy with fewer side effects than treatment with either single agent at the monotherapy clinical dose. As a result, combination therapy is more likely to achieve the desired medical benefits without the trial and error involved in prescribing each agent individually during primary care.

### SUMMARY OF THE INVENTION

The present invention provides compositions comprising an anti-obesity agent which is a particular cannabinoid CB₁ receptor antagonist/inverse agonist and an anti-diabetic agent which is a particular DPP-IV inhibitor, which compositions are useful in the treatment, control and/or prevention of diabetes, diabetes associated with obesity, diabetes-related disorders, obesity, and obesity-related disorders.

The therapeutic utility of DPP-IV inhibitors for the treatment of Type 2 diabetes is discussed by (i) D. J. Drucker in Exp. Opin. Invest. Drugs. 12: 87-100 (2003); (ii) K. Augustyns, et al., in Exp. Opin. Ther. Patents, 13: 499-510 (2003); (iii) C. F. Deacon, et al., in Exp. Opin. Investig. Drugs, 13: 1091-1102 (2004); (iv) A. E. Weber, “Dipeptidyl Peptidase IV Inhibitors for the Treatment of Diabetes,” J. Med. Chem., 47: 4135-4141 (2004); and (v) J. J. Holst, “Treatment of Type 2 diabetes mellitus with agonists of the GLP-1 receptor or DPP-IV inhibitors,” Exp. Opin. Emerg. Drugs, 9: 155-166 (2004).

The therapeutic effects of cannabinoid receptor ligands has been described in (i) R. G. Pertwee, “Cannabinoid receptor ligands: clinical and neuropharmacological considerations, relevant to future drug discovery and development, Exp. Opin. Invest. Drugs, 9: 1553-1571 (2000) and (ii) A. J. Drysdale, “Cannibinoids: Mechanisms and Therapeutic Applications in the CNS,” Curr. Med. Chem., 10: 2719-2732 (2003).

The compositions of the present invention are useful in the treatment, control and/or prevention of diabetes, in particular Type 2 diabetes, in humans.

The compositions of the present invention are further useful in the treatment, control and/or prevention of hyperlipidemia; dyslipidemia; obesity; abdominal obesity; hypercholesterolemia; hypertrigyceridemia; atherosclerosis; coronary heart disease; stroke; hypertension; peripheral vascular disease; vascular restenosis; nephropathy; neuropathy; inflammatory conditions, such as, but not limited to, irritable bowel syndrome, inflammatory bowel disease, including Crohn's disease and ulcerative colitis; other inflammatory conditions; pancreatitis; neurodegenerative disease; retinopathy; neoplastic conditions, such as, but not limited to adipose cell tumors, adipose cell carcinomas, such as liposarcoma; cancers, including gastric and bladder cancers; angiogenesis; Alzheimer's disease; psoriasis; and other disorders where insulin resistance is a component.

The compositions of the present invention are also useful in the treatment, control and/or prevention of overeating; bulimia; elevated plasma insulin concentrations; insulin resistance; glucose tolerance; lipid disorders; low HDL levels; high LDL levels; hyperglycemia; neoplastic conditions, such as endometrial, breast, prostate, kidney and colon cancer; osteoarthritis; obstructive sleep apnea; gallstones; abnormal heart rhythms; heart arrythmias; myocardial infarction; congestive heart failure; sudden death; ovarian hyperandrogenism, (polycystic ovary disease); craniopharyngioma; the Prader-Willi Syndrome; Frohlich's syndrome; GH-deficient subjects; normal variant short stature; Turner's syndrome; and other pathological conditions showing reduced metabolic activity or a decrease in resting energy expenditure as a percentage of total fat-free mass, e.g, children with acute lymphoblastic leukemia.

The compositions of the present invention are also useful in the treatment, control, and/or prevention of diabetes while mitigating cardiac hypertrophy, including left ventricular hypertrophy.

The compositions of the present invention are further useful in the treatment, control, and/or prevention of Metabolic Syndrome.

The present invention is also concerned with treatment of these conditions, and the use of the compositions of the present invention for the manufacture of a medicament useful for treating these conditions.

The invention is also concerned with pharmaceutical compositions comprising a particular DPP-IV inhibitor and a particular cannabinoid CB₁ receptor antagonist/inverse agonist as active pharmaceutical ingredients.

The present invention is also concerned with the use of a particular DPP-IV inhibitor and a particular cannabinoid CB₁ receptor antagonist/inverse agonist, for the manufacture of a medicament for the treatment of diabetes, diabetes associated with obesity, diabetes-related disorders, obesity, and obesity-related disorders, which comprises a therapeutically effective amount of such DPP-IV inhibitor and such cannabinoid CB₁ receptor antagonist/inverse agonist, together or separately.

The present invention is also concerned with a drug product containing a particular DPP-IV inhibitor and a particular cannabinoid CB₁ receptor antagonist/inverse agonist, as a combined preparation for simultaneous, separate or sequential use in diabetes, diabetes associated with obesity, diabetes-related disorders, obesity, and obesity-related disorders.

The present invention also relates to the treatment of diabetes, diabetes associated with obesity, diabetes-related disorders, obesity, and obesity-related disorders with a combination of a particular DPP-IV inhibitor and a particular cannabinoid CB₁ receptor antagonist/inverse agonist, which may be administered separately.

The invention also relates to combining separate pharmaceutical combinations into a kit form.

### DETAILED DESCRIPTION OF THE INVENTION

The present invention provides compositions comprising an anti-obesity agent which is a particular cannabinoid CB₁ receptor antagonist/inverse agonist and an anti-diabetic agent which is a particular DPP-IV inhibitor which compositions are useful in the treatment or prevention of diabetes, diabetes associated with obesity, diabetes-related disorders, obesity, and obesity-related disorders.

The cannabinoid CB₁ receptor antagonist/inverse agonists used in the compositions of the present invention are selected from the group consisting of:
N-[3-(4-chlorophenyl)-1-methyl-2-phenylpropyl]-2-(2-pyridyloxy)-2-methylpropanamide;
N-[3-(4-chlorophenyl)-2-(3,5-difluorophenyl)-1-methylpropyl]-2-(2-pyridyloxy)-2-methylpropanamide;
N-[3-(4-chlorophenyl)-1-methyl-2-phenyl-propyl]-2-(5-chloro-2-pyridyloxy)-2-methylpropanamide;
N-[3-(4-chlorophenyl)-2-(3-pyridyl)-1-methylpropyl]-2-(5-trifluoromethyl-2-pyridyloxy)-2-methylpropanamide;
N-[3-(4-chlorophenyl)-2-(3-cyanophenyl)-1-methylpropyl]-2-(5-trifluoromethyl-2-pyridyloxy)-2-methylpropanamide;
N-[3-(4-chlorophenyl)-2-(5-chloro-3-pyridyl)-1-methylpropyl]-2-(5-trifluoromethyl-2-pyridyloxy)-2-methylpropanamide;
N-[3-(4-chlorophenyl)-2-(5-methyl-3-pyridyl)-1-methylpropyl]-2-(5-trifluoromethyl-2-pyridyloxy)-2-methylpropanamide;
N-[3-(4-chlorophenyl)-2-(5-cyano-3-pyridyl)-1-methylpropyl]-2-(5-trifluoromethyl-2-pyridyloxy)-2-methylpropanamide;
N-[3-(4-chlorophenyl)-2-(3-methylphenyl)-1-methylpropyl]-2-(5-trifluoromethyl-2-pyridyloxy)-2-methylpropanamide;
N-[3-(4-chlorophenyl)-2-phenyl-1-methylpropyl]-2-(4-trifluoromethyl-2-pyridyloxy)-2-methylpropanamide;
N-[3-(4-chlorophenyl)-2-phenyl-1-methylpropyl]-2-(4-trifluoromethyl-2-pyrimidyloxy)-2-methylpropanamide;
N-[3-(4-chlorophenyl)-1-methyl-2-(thiophen-3-yl)propyl]-2-(5-chloro-2-pyridyloxy)-2-methylpropanamide;
N-[3-(5-chloro-2-pyridyl)-2-phenyl-1-methylpropyl]-2-(5-trifluoromethyl-2-pyridyloxy)-2-methylpropanamide;
N-[3-(4-fluorophenyl)-2-(3-cyanophenyl)-1-methylpropyl]-2-(5-trifluoromethyl-2-pyridyloxy)-2-methylpropanamide;
N-[3-(4-methoxy-phenyl)-2-(3-cyanophenyl)-1-methylpropyl]-2-(5-trifluoromethyl-2-pyridyloxy)-2-methylpropanamide;
N-[3-(4-chlorophenyl)-2-(3-cyanophenyl)-1-methylpropyl]-2-(6-trifluoromethyl-4-pyrimidyloxy)-2-methylpropanamide;
N-[2-(3-cyanophenyl)-1,4-dimethylpentyl]-2-(5-trifluoromethyl-2-pyridyloxy)-2-methylpropanamide;
N-[2-(3-cyanophenyl)-3-cyclobutyl-1-methylpropyl]-2-(5-trifluoromethyl-2-pyridyloxy)-2-methylpropanamide;
N-[2-(3-cyanophenyl)-1-methyl-heptyl]-2-(5-trifluoromethyl-2-pyridyloxy)-2-methylpropanamide;
N-[2-(3-cyanophenyl)-3-cyclopentyl-1-methylpropyl]-2-(5-trifluoromethyl-2-pyridyloxy)-2-methylpropanamide;
N-[2-(3-cyanophenyl)-3-cyclohexyl-1-methylpropyl]-2-(5-trifluoromethyl-2-pyridyloxy)-2-methylpropanamide;
3-{1-[bis(4-chlorophenyl)methyl]azetidin-3-ylidene}-3-(3,5-difluorophenyl)-2,2-dimethylpropanenitrile;
1-{1-[1-(4-chlorophenyl)pentyl]azetidin-3-yl}-1-(3,5-difluorophenyl)-2-methylpropan-2-ol;
3-((S)-(4-chlorophenyl){3-[(1S)-1-(3,5-difluorophenyl)-2-hydroxy-2-methylpropyl]azetidin-1-yl}methyl)benzonitrile;
3-((S)-(4-chlorophenyl){3-[(1S)-1-(3,5-difluorophenyl)-2-fluoro-2-methylpropyl]azetidin-1-yl}methyl)benzonitrile;
3-((4-chlorophenyl){3-[1-(3,5-difluorophenyl)-2,2-dimethylpropyl]azetidin-1-yl}methyl)benzonitrile;
3-((1S)-1-{1-[(S)-3-cyanophenyl)(4-cyanophenyl)methyl]azetidin-3-yl}-2-fluoro-2-methylpropyl)-5-fluorobenzonitrile;
3-[(S)-(4-chlorophenyl)(3-{(1S)-2-fluoro-1-[3-fluoro-5-4H-1,2,4-triazol-4-yl)phenyl]-2-methylpropyl}azetidin-1-yl)methyl]benzonitrile; and
5-((4-chlorophenyl){3-[(1S)-1-(3,5-difluorophenyl)-2-fluoro-2-methylpropyl]azetidin-1-yl}methyl)thiophene-3-carbonitrile;
   and stereoisomers, pharmaceutically acceptable salts, hydrates, and crystalline forms thereof.

The above cannabinoid CB₁ receptor antagonist/inverse agonists are disclosed in international patent publication WO 2003/077847 (published 24 Sep. 2003) and international patent publication WO 2005/000809 (published 6 Jan. 2005).

In one embodiment the cannabinoid CB₁ receptor antagonist/inverse agonist is N-[(1S,2S)-3-(4-chlorophenyl)-2-(3-cyanophenyl)-1-methylpropyl]-2-methyl-{[5-(trifluoromethyl)pyridin-2-yl]oxy}propanamide of structural formula III:

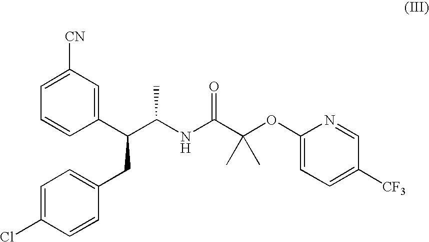

or a pharmaceutically acceptable salt, hydrate, or crystalline form thereof.

The methods and compositions of the present invention also comprise an anti-diabetic agent which is a particular DPP-IV inhibitor selected from the group consisting of:

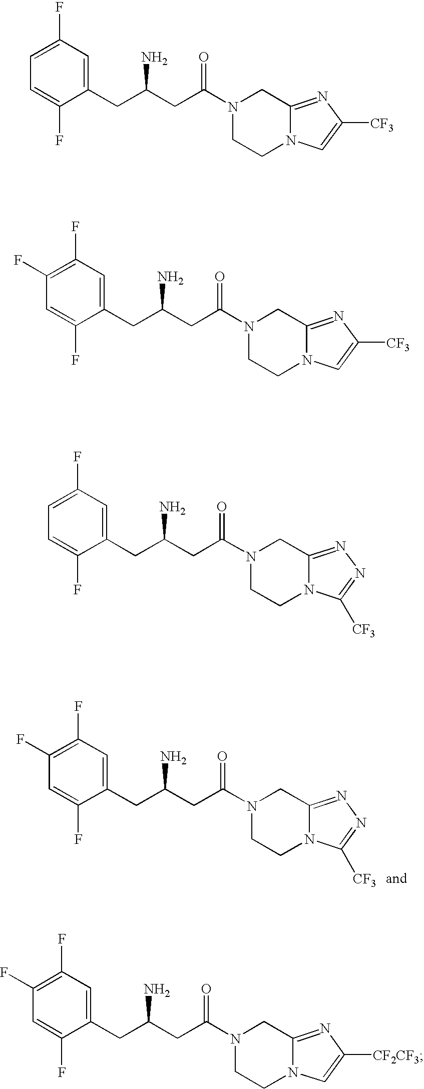

and pharmaceutically acceptable salts, hydrates, and crystalline forms thereof.

In one embodiment the particular DPP-IV inhibitor is (2R)-4-oxo-4-[3-(trifluoromethyl)-5,6-dihydro[1,2,4]triazolo[4,3α]pyrazin-7(8H)-yl]-1-(2,4,5-trifluorophenyl)butan-2-amine of structural formula IV:

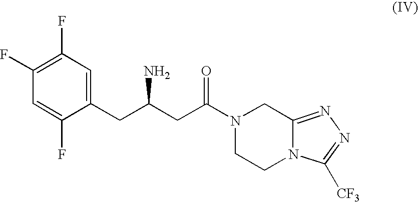

or a pharmaceutically acceptable salt, hydrate, or crystalline form thereof.

In a class of this embodiment the pharmaceutically acceptable salt of the compound of formula IV is the dihydrogenphosphate salt of structural formula V:

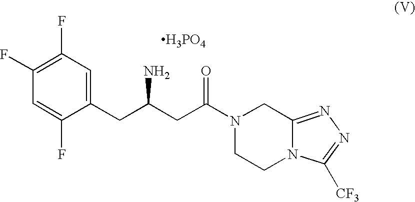

or a pharmaceutically acceptable hydrate thereof. In a subclass of this class the pharmaceutically acceptable hydrate is a crystalline monohydrate. The crystalline dihydrogenphosphate monohydrate is referred to as MK-0431.

The DPP-IV inhibitors in the compositions of the present invention are disclosed in U.S. Pat. No. 6,699,871 (Mar. 2, 2004), the contents of which are incorporated herein by reference in their entirety. The development of this series of DPP-IV inhibitors has been described by D. Kim, et al., in J. Med. Chem., 48: 141-151 (2005).

The present invention also relates to a method of treating, controlling, or preventing diabetes, particularly non-insulin dependent diabetes mellitus, comprising administering to a subject in need thereof:

(a) a therapeutically or prophylactically effective amount of a particular cannabinoid CB₁ receptor antagonist/inverse agonist, or a pharmaceutically acceptable salt thereof; and
(b) a therapeutically or prophylactically effective amount of a particular DPP-IV inhibitor, or a pharmaceutically acceptable salt thereof.

The present invention relates to a method of treating, controlling, or preventing diabetes associated with obesity comprising administering to a subject in need thereof:

(a) a therapeutically or prophylactically effective amount of a particular cannabinoid CB₁ receptor antagonist/inverse agonist, or a pharmaceutically acceptable salt thereof; and
(b) a therapeutically or prophylactically effective amount of a particular DPP-IV inhibitor, or a pharmaceutically acceptable salt thereof.

The present invention relates to a method of treating, controlling, or preventing hypercholesterolemia, atherosclerosis, low HDL levels, high LDL levels, hyperlipidemia, hypertriglyceridemia, and/or dyslipidemia, comprising administering to a subject in need thereof:

(a) a therapeutically or prophylactically effective amount of a particular cannabinoid CB₁ receptor antagonist/inverse agonist, or a pharmaceutically acceptable salt thereof; and
(b) a therapeutically or prophylactically effective amount of a particular DPP-IV inhibitor, or a pharmaceutically acceptable salt thereof.

The present invention relates to a method of treating, controlling, or preventing hyperglycemia comprising administering to a subject in need thereof:

(a) a therapeutically or prophylactically effective amount of a particular cannabinoid CB₁ receptor antagonist/inverse agonist, or a pharmaceutically acceptable salt thereof; and
(b) a therapeutically or prophylactically effective amount of a particular DPP-IV inhibitor, or a pharmaceutically acceptable salt thereof.

The present invention relates to a method of treating, controlling, or preventing hypercholesterolemia comprising administering to a subject in need thereof:

(a) a therapeutically or prophylactically effective amount of a particular cannabinoid CB₁ receptor antagonist/inverse agonist, or a pharmaceutically acceptable salt thereof; and
(b) a therapeutically or prophylactically effective amount of a particular DPP-IV inhibitor, or a pharmaceutically acceptable salt thereof.

The present invention relates to a method of treating, controlling, or preventing hypertriglyceridemia comprising administering to a subject in need thereof:

(a) a therapeutically or prophylactically effective amount of a particular cannabinoid CB₁ receptor antagonist/inverse agonist, or a pharmaceutically acceptable salt thereof; and
(b) a therapeutically or prophylactically effective amount of a particular DPP-IV inhibitor, or a pharmaceutically acceptable salt thereof.

The present invention relates to a method of treating, controlling, or preventing lipid disorders, hyperlipidemia, dyslipidemia, high non-HDL cholesterol, and low-HDL cholesterol comprising administering to a subject in need thereof:

(a) a therapeutically or prophylactically effective amount of a particular cannabinoid CB₁ receptor antagonist/inverse agonist, or a pharmaceutically acceptable salt thereof; and
(b) a therapeutically or prophylactically effective amount of a particular DPP-IV inhibitor, or a pharmaceutically acceptable salt thereof.

The present invention relates to a method of treating, controlling, or preventing dyslipidemia, including low HDL cholesterol comprising administering to a subject in need thereof:

(a) a therapeutically or prophylactically effective amount of a particular cannabinoid CB₁ receptor antagonist/inverse agonist, or a pharmaceutically acceptable salt thereof; and
(b) a therapeutically or prophylactically effective amount of a particular DPP-IV inhibitor, or a pharmaceutically acceptable salt thereof.

The present invention relates to a method of treating, controlling, or preventing atherosclerosis comprising administering to a subject in need thereof:

(a) a therapeutically or prophylactically effective amount of a particular cannabinoid CB₁ receptor antagonist/inverse agonist, or a pharmaceutically acceptable salt thereof; and
(b) a therapeutically or prophylactically effective amount of a particular DPP-IV inhibitor, or a pharmaceutically acceptable salt thereof.

It is understood that the sequellae of atherosclerosis (angina, claudication, heart attack, stroke, etc.) are thereby treated.

The present invention relates to a method of treating, controlling, or preventing diabetes while mitigating cardiac hypertrophy, particularly left ventricular hypertrophy, comprising administering to a subject in need thereof:

(a) a therapeutically or prophylactically effective amount of a particular cannabinoid CB₁ receptor antagonist/inverse agonist, or a pharmaceutically acceptable salt thereof; and
(b) a therapeutically or prophylactically effective amount of a particular DPP-IV inhibitor, or a pharmaceutically acceptable salt thereof.

The present invention relates to a method of treating, controlling, or preventing Metabolic Syndrome comprising administering to a subject in need thereof:

(a) a therapeutically or prophylactically effective amount of a particular cannabinoid CB₁ receptor antagonist/inverse agonist, or a pharmaceutically acceptable salt thereof; and
(b) a therapeutically or prophylactically effective amount of a particular DPP-IV inhibitor, or a pharmaceutically acceptable salt thereof.

The present invention relates to a method of treating a diabetes-related disorder comprising administering to a subject in need thereof:

(a) a therapeutically effective amount of a particular cannabinoid CB₁ receptor antagonist/inverse agonist, or a pharmaceutically acceptable salt thereof; and
(b) a therapeutically effective amount of a particular DPP-IV inhibitor, or a pharmaceutically acceptable salt thereof.

The present invention relates to a method of treating or preventing obesity comprising administering to a subject in need thereof:

(a) a therapeutically or prophylactically effective amount of a particular cannabinoid CB₁ receptor antagonist/inverse agonist, or a pharmaceutically acceptable salt thereof; and
(b) a therapeutically or prophylactically effective amount of a particular DPP-IV inhibitor, or a pharmaceutically acceptable salt thereof.

The present invention relates to a method of treating or preventing an obesity-related disorder comprising administering to a subject in need thereof:

(a) a therapeutically or prophylactically effective amount of a particular cannabinoid CB₁ receptor antagonist/inverse agonist, or a pharmaceutically acceptable salt thereof; and
(b) a therapeutically or prophylactically effective amount of a particular DPP-IV inhibitor, or a pharmaceutically acceptable salt thereof.

The present invention also relates to pharmaceutical compositions and medicaments useful for carrying out these methods.

The present invention relates to the use of an anti-obesity agent which is a particular cannabinoid CB₁ receptor antagonist/inverse agonist, or a pharmaceutically acceptable salt, hydrate, or crystalline form thereof; and an anti-diabetic agent which is a particular DPP-IV inhibitor, or a pharmaceutically acceptable salt, hydrate, or crystalline form thereof; for the manufacture of a medicament for treatment, control, or prevention of diabetes which comprises an effective amount of such anti-obesity agent and an effective amount of such anti-diabetic agent, together or separately.

The present invention also relates to the use of an anti-obesity agent which is a particular cannabinoid CB₁ receptor antagonist/inverse agonist, or a pharmaceutically acceptable salt, hydrate, or crystalline form thereof; and an anti-diabetic agent which is a particular DPP-IV inhibitor, or a pharmaceutically acceptable salt, hydrate, or crystalline form thereof; for the manufacture of a medicament for treatment, control, or prevention of diabetes associated with obesity which comprises a therapeutically or prophylactically effective amount of such anti-obesity agent and an effective amount of such anti-diabetic agent, together or separately.

The present invention relates to the use of an anti-obesity agent which is a particular cannabinoid CB₁ receptor antagonist/inverse agonist, or a pharmaceutically acceptable salt, hydrate, or crystalline form thereof; and an anti-diabetic agent which is a particular DPP-IV inhibitor, or a pharmaceutically acceptable salt, hydrate, or crystalline form thereof; for the manufacture of a medicament for treatment, control, or prevention of a diabetes-related disorder which comprises a therapeutically or prophylactically effective amount of such anti-obesity agent and an effective amount of such anti-diabetic agent, together or separately.

The present invention relates to the use of an anti-obesity agent which is a particular cannabinoid CB₁ receptor antagonist/inverse agonist, or a pharmaceutically acceptable salt, hydrate, or crystalline form thereof; and an anti-diabetic agent which is a particular DPP-IV inhibitor, or a pharmaceutically acceptable salt, hydrate, or crystalline form thereof; for the manufacture of a medicament for treatment, control, or prevention of obesity which comprises a therapeutically or prophylactically effective amount of such anti-obesity agent and an effective amount of such anti-diabetic agent, together or separately.

The present invention relates to the use of an anti-obesity agent which is a particular cannabinoid CB₁ receptor antagonist/inverse agonist, or a pharmaceutically acceptable salt, hydrate, or crystalline form thereof; and an anti-diabetic agent which is a particular DPP-IV inhibitor, or a pharmaceutically acceptable salt, hydrate, or crystalline form thereof; for the manufacture of a medicament for treatment, control, or prevention of an obesity-related disorder which comprises a therapeutically or prophylactically effective amount of such anti-obesity agent and an effective amount of such anti-diabetic agent, together or separately.

The present invention relates to the use of an anti-obesity agent which is a particular cannabinoid CB₁ receptor antagonist/inverse agonist, or a pharmaceutically acceptable salt, hydrate, or crystalline form thereof; and an anti-diabetic agent which is a particular DPP-IV inhibitor, or a pharmaceutically acceptable salt, hydrate, or crystalline form thereof; for the manufacture of a medicament for treatment, control, or prevention of Metabolic Syndrome which comprises a therapeutically or prophylactically effective amount of such anti-obesity agent and an effective amount of such anti-diabetic agent, together or separately.

The present invention relates to the use of an anti-obesity agent which is a particular cannabinoid CB₁ receptor antagonist/inverse agonist, or a pharmaceutically acceptable salt, hydrate, or crystalline form thereof; and an anti-diabetic agent which is a particular DPP-IV inhibitor, or a pharmaceutically acceptable salt, hydrate, or crystalline form thereof; for the manufacture of a medicament for treatment, control, or prevention of diabetes while mitigating cardiac hypertrophy, particularly left ventricular hypertrophy, which comprises a therapeutically or prophylactically effective amount of such anti-obesity agent and a therapeutically or prophylactically effective amount of such anti-diabetic agent, together or separately.

The present invention further relates to a drug product containing an anti-obesity agent which is a particular cannabinoid CB₁ receptor antagonist/inverse agonist, or a pharmaceutically acceptable salt, hydrate, or crystalline form thereof; and an anti-diabetic agent which is a particular DPP-IV inhibitor, or a pharmaceutically acceptable salt, hydrate, or crystalline form thereof; as a combined preparation for simultaneous, separate or sequential use in diabetes.

The present invention further relates to a drug product containing an anti-obesity agent which is a particular cannabinoid CB₁ receptor antagonist/inverse agonist, or a pharmaceutically acceptable salt, hydrate, or crystalline form thereof; and an anti-diabetic agent which is a particular DPP-IV inhibitor, or a pharmaceutically acceptable salt, hydrate, or crystalline form thereof; as a combined preparation for simultaneous, separate or sequential use in a diabetes-related disorder.

The present invention further relates to a drug product containing an anti-obesity agent which is a particular cannabinoid CB₁ receptor antagonist/inverse agonist, or a pharmaceutically acceptable salt, hydrate, or crystalline form thereof; and an anti-diabetic agent which is a particular DPP-IV inhibitor, or a pharmaceutically acceptable salt, hydrate, or crystalline form thereof; as a combined preparation for simultaneous, separate or sequential use in obesity.

The present invention further relates to a drug product containing an anti-obesity agent which is a particular cannabinoid CB₁ receptor antagonist/inverse agonist, or a pharmaceutically acceptable salt, hydrate, or crystalline form thereof; and an anti-diabetic agent which is a particular DPP-IV inhibitor, or a pharmaceutically acceptable salt, hydrate, or crystalline form thereof; as a combined preparation for simultaneous, separate or sequential use in an obesity-related disorder.

The present invention further relates to a drug product containing an anti-obesity agent which is a particular cannabinoid CB₁ receptor antagonist/inverse agonist, or a pharmaceutically acceptable salt, hydrate, or crystalline form thereof; and an anti-diabetic agent which is a particular DPP-IV inhibitor, or a pharmaceutically acceptable salt, hydrate, or crystalline form thereof; as a combined preparation for simultaneous, separate or sequential use in Metabolic Syndrome.

The present invention further relates to a drug product containing an anti-obesity agent which is a particular cannabinoid CB₁ receptor antagonist/inverse agonist, or a pharmaceutically acceptable salt, hydrate, or crystalline form thereof; and an anti-diabetic agent which is a particular DPP-IV inhibitor, or a pharmaceutically acceptable salt, hydrate, or crystalline form thereof; as a combined preparation for simultaneous, separate or sequential use in diabetes while mitigating cardiac hypertrophy, particularly left ventricular hypertrophy.

The invention further provides pharmaceutical compositions comprising an anti-obesity agent which is a particular cannabinoid CB₁ receptor antagonist/inverse agonist, or a pharmaceutically acceptable salt, hydrate, or crystalline form thereof; and an anti-diabetic agent which is a particular DPP-IV inhibitor, or a pharmaceutically acceptable salt, hydrate, or crystalline form thereof, as active ingredients.

The present invention further relates to the treatment or prevention of diabetes, diabetes associated with obesity, a diabetes-related disorder, obesity or an obesity-related disorder with a combination of an anti-obesity agent which is a particular cannabinoid CB₁ receptor antagonist/inverse agonist and an anti-diabetic agent which is a particular DPP-IV inhibitor, which may be administered separately. Therefore the invention also relates to combining separate pharmaceutical compositions into a kit form. The kit, according to this invention, comprises two separate pharmaceutical compositions: a first unit dosage form comprising a prophylactically or therapeutically effective amount of a particular cannabinoid CB₁ receptor antagonist/inverse agonist, or a pharmaceutically acceptable salt, hydrate, or crystalline form thereof, and a pharmaceutically acceptable carrier or diluent in a first unit dosage form, and a second unit dosage form comprising a prophylactically or therapeutically effective amount of a particular DPP-IV inhibitor, or a pharmaceutically acceptable salt, hydrate, or crystalline form thereof, and a pharmaceutically acceptable carrier or diluent in a second unit dosage form.

The present invention also relates to a kit comprising at least one unit dosage of a prophylactically or therapeutically effective amount of a particular cannabinoid CB₁ receptor antagonist/inverse agonist, or a pharmaceutically acceptable salt, hydrate, or crystalline form thereof, and at least one unit dosage of a prophylactically or therapeutically effective amount of a particular DPP-IV inhibitor, or a pharmaceutically acceptable salt, hydrate, or crystalline form thereof.

In one embodiment of the present invention, the kit further comprises a container. Such kits are especially suited for the delivery of solid oral forms such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days or time in the treatment schedule in which the dosages can be administered.

### Combination Therapy

The compositions of the present invention may be used in combination with other drugs that may also be useful in the treatment, prevention, or control of diabetes, diabetes associated with obesity, diabetes-related disorders, obesity, and obesity-related disorders for which compounds comprising the compositions are useful. Such other drugs may be administered, by a route and in an amount commonly used therefore, contemporaneously or sequentially with a composition of the present invention. When a composition of the present invention is used contemporaneously with one or more other drugs, a pharmaceutical composition in unit dosage form containing such other drugs and the composition of the present invention is preferred. However, the combination therapy also includes therapies in which the composition of the present invention and one or more other drugs are administered on different overlapping schedules. It is also contemplated that when used in combination with one or more other active ingredients, the composition of the present invention and the other active ingredients may be used in lower doses than when each is used singly. Accordingly, the pharmaceutical compositions of the present invention include those that contain one or more other active ingredients, in addition to a composition of the present invention.

Examples of other active ingredients that may be administered in combination with a composition of the present invention, and either administered separately or in the same pharmaceutical composition, include, but are not limited to:

(a) insulin sensitizers including (i) PPARα agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, tesaglitazar, and TAK-559; PPARα agonists, such as fenofibric acid derivatives (gemfibrozil, clofibrate, fenofibrate and bezafibrate); and selective PPARγ modulators (SPPARγM's), such as disclosed in WO 02/060388, WO 02/08188, WO 2004/019869, WO 2004/020409, WO 2004/020408, and WO 2004/066963; (ii) biguanides, such as metformin and phenformin, and (iii) protein tyrosine phosphatase-1B (PTP-1B) inhibitors;

(b) insulin or insulin mimetics;

(c) sulfonylureas and other insulin secretagogues, such as tolbutamide, glyburide, glipizide, glimepiride, and meglitinides, such as nateglinide and repaglinide;

(d) α-glucosidase inhibitors (such as acarbose and miglitol);

(e) glucagon receptor antagonists, such as those disclosed in WO 97/16442; WO 98/04528, WO 98/21957; WO 98/22108; WO 98/22109; WO 99/01423, WO 00/39088, and WO 00/69810; WO 2004/050039; and WO 2004/069158;

(f) GLP-1, GLP-1 analogues or mimetics, and GLP-1 receptor agonists, such as exendin-4 (exenatide), liraglutide (N,N-2211), CJC-1131, LY-307161, and those disclosed in WO 00/42026 and WO 00/59887;

(g) GIP and GIP mimetics, such as those disclosed in WO 00/58360, and GIP receptor agonists;

(h) PACAP, PACAP mimetics, and PACAP receptor agonists such as those disclosed in WO 01/23420;

(i) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists, such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(j) PPARδ agonists, such as those disclosed in WO 97/28149;

(k) antiobesity compounds, such as fenfluramine, dexfenfluramine, phentermine, sibutramine, orlistat, neuropeptide Y₁ or Y₅ antagonists, β₃ adrenergic receptor agonists, melanocortin-receptor agonists, in particular melanocortin-4 receptor agonists, ghrelin antagonists, bombesin receptor agonists (such as bombesin receptor subtype-3 agonists), and melanin-concentrating hormone (MCH) receptor antagonists;

(l) ileal bile acid transporter inhibitors;

(m) agents intended for use in inflammatory conditions such as aspirin, non-steroidal anti-inflammatory drugs NSAIDs), glucocorticoids, azulfidine, and selective cyclooxygenase-2 (COX-2) inhibitors;

(n) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(o) glucokinase activators (GKAs), such as those disclosed in WO 03/015774; WO 04/076420; and WO 04/081001;

(p) inhibitors of 11β-hydroxysteroid dehydrogenase Type 1, such as those disclosed in U.S. Pat. No. 6,730,690; WO 03/104207; and WO 04/058741;

(q) inhibitors of cholesteryl ester transfer protein (CETP), such as torcetrapib; and

(r) inhibitors of fructose 1,6-bisphosphatase, such as those disclosed in U.S. Pat. Nos. 6,054,587; 6,110,903; 6,284,748; 6,399,782; and 6,489,476.

The above combinations include combinations of a composition of the present invention not only with one other active compound, but also with two or more other active compounds. Non-limiting examples include combinations of the compositions of the present invention with one, two or more active compounds selected from anti-dyslipidemic agents and anti-hypertensive agents. Combinations of the compositions of the present invention with one, two, or more active compounds selected from anti-dyslipidemic agents and anti-hypertensive agents will be useful to treat, control or prevent Metabolic Syndrome. Combinations of the compositions of the present invention comprising a DPP-IV inhibitor and a cannabinoid CB₁ receptor antagonist/inverse agonist, in addition to an anti-dyslipidemic agent and/or an anti-hypertensive agent are more efficacious in the treatment, control, or prevention of Metabolic Syndrome than treatment with any of these agents alone. In particular, compositions comprising a DPP-IV inhibitor and a cannabinoid CB₁ receptor antagonist/inverse agonist, an anti-hypertensive agent and/or an anti-dyslipidemic agent will be useful to synergistically treat, control or prevent Metabolic Syndrome.

### DEFINITIONS

The compounds in the compositions of the present invention include stereoisomers, such as optical isomers and diastereomers depending on the mode of substitution. The compounds may contain one or more chiral centers and occur as racemates, racemic mixtures and as individual diastereomers, enantiomeric mixtures or single enantiomers, with all isomeric forms being included in the present invention. The present invention is meant to comprehend all such isomeric forms of the compounds in the compositions of the present invention, and their mixtures. Therefore, where a compound is chiral, the separate enantiomers, substantially free of the other, are included within the scope of the invention; further included are all mixtures of the two enantiomers. Also included within the scope of the invention are crystalline forms and hydrates, such as hydrates, of the compounds of the instant invention.

The independent syntheses of these stereoisomers or their chromatographic separations may be achieved as known in the art by appropriate modification of the methodology disclosed herein. Their absolute stereochemistry may be determined by the X-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

The present invention includes within its scope prodrugs of the compounds in the compositions of this invention. In general, such prodrugs will be functional derivatives of the compounds in these compositions which are readily convertible in vivo into the required compound. Thus, in the methods of treatment of the present invention, the term “administering” shall encompass the treatment of diabetes, diabetes associated with obesity, diabetes-related disorders, obesity, and obesity-related disorders with the compounds specifically disclosed as elements of the composition or with compounds which may not be specifically disclosed, but which convert to the specified compounds in vivo after administration to the patient. Conventional procedures for the selection and preparation of suitable prodrug derivatives are described, for example, in “Design of Prodrugs,” ed. H. Bundgaard, Elsevier, 1985.

Some of the compounds described herein contain olefinic double bonds, and unless specified otherwise, are meant to include both E and Z geometric isomers.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

The pharmaceutically acceptable salts of the composition of the instant invention include the composition wherein one of the individual components of the composition is in the form of a pharmaceutically acceptable salt, or the composition wherein all of the individual components are in the form of pharmaceutically acceptable salts (wherein the salts for each of the components can be the same or different), or a pharmaceutically acceptable salt of the combined components (i.e., a salt of the composition).

The term “composition” as used herein is intended to encompass a product comprising the specified ingredients in the specified amounts, as well as any product which results, directly or indirectly, from combination of the specified ingredients in the specified amounts. Such term, in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

### Utilities:

The compositions of the present invention are useful for the treatment or prevention of diabetes, and especially non-insulin dependent diabetes mellitus (NIDDM). The diabetes herein may be due to any cause, whether genetic or environmental.

The compositions of the present invention are useful for the treatment or prevention of diabetes associated with obesity. Diabetes associated with obesity may be associated with, caused by, or result from obesity.

The compositions of the present invention are useful for the treatment or prevention of diabetes-related disorders. The diabetes-related disorders herein are associated with, caused by, or result from diabetes. Examples of diabetes-related disorders include hyperglycemia, impaired glucose tolerance, insulin resistance, obesity, lipid disorders, dyslipidemia, hyperlipidemia, hypertriglyceridemia, hypercholesterolemia, low HDL levels, high LDL levels, atherosclerosis and its sequelae, vascular restenosis, irritable bowel syndrome, inflamatory bowel disease, including Crohn's disease and ulcerative colitis, other inflammatory conditions, pancreatitis, abdominal obesity, neurodegenerative disease, retinopathy, neoplastic conditions, adipose cell tumors, adipose cell carcinomas, such as liposarcoma, prostate cancer and other cancers, including gastric, breast, bladder and colon cancers, angiogenesis, Alzheimer's disease, psoriasis, high blood pressure, Metabolic Syndrome, ovarian hyperandrogenism (polycystic ovary syndrome), and other disorders where insulin resistance is a component, such as sleep apnea. The compositions of the present invention are particularly useful for the treatment, control or prevention of hyperglycemia, impaired glucose tolerance, obesity, dyslipidemia, hyperlipidemia, hypertriglyceridemia, hypercholesterolemia, low HDL levels, atherosclerosis, and Metabolic Syndrome.

The compositions of the present invention are also useful for the treatment or prevention of Metabolic Syndrome. The compositions of the present invention further comprising an anti-hypertensive agent and/or an anti-dyslipidemic agent are useful for the treatment or prevention of Metabolic Syndrome.

The compositions of the present invention are useful for the treatment or prevention of diabetes.

The compositions of the present invention are useful for the treatment, control, or prevention of obesity. The obesity herein may be due to any cause, whether genetic or environmental. The compositions comprised of a DPP-IV inhibitor and a cannabinoid CB₁ receptor antagonist/inverse agonist are also useful to treat and/or prevent the weight gain associated with treatment with certain anti-diabetic agents, such as PPAR-γ agonists.

The compositions of the present invention are useful for the treatment, control, or prevention of obesity-related disorders. The obesity-related disorders herein are associated with, caused by, or result from obesity. Examples of obesity-related disorders include obesity, diabetes, overeating, binge eating, and bulimia, hypertension, elevated plasma insulin concentrations and insulin resistance, dyslipidemia, hyperlipidemia, endometrial, breast, prostate, kidney and colon cancer, osteoarthritis, obstructive sleep apnea, gallstones, heart disease, abnormal heart rhythms and arrythmias, myocardial infarction, congestive heart failure, coronary heart disease, sudden death, stroke, polycystic ovarian syndrome, craniopharyngioma, Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are Metabolic Syndrome, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-esophageal reflux, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, lower back pain, gallbladder disease, hyperuricemia, gout, and kidney cancer, and increased anesthetic risk. The compositions of the present invention are also useful to treat Alzheimer's disease and smoking.

The term “diabetes,” as used herein, includes both insulin-dependent diabetes mellitus (i.e., IDDM, also known as Type 1 diabetes) and non-insulin-dependent diabetes mellitus (i.e., NIDDM, also known as Type 2 diabetes). Type 1 diabetes, or insulin-dependent diabetes, is the result of an absolute deficiency of insulin, the hormone which regulates glucose utilization. Type 2 diabetes, or insulin-independent diabetes (i.e., non-insulin-dependent diabetes mellitus), often occurs in the face of normal, or even elevated levels of insulin and appears to be the result of the inability of tissues to respond appropriately to insulin. The development of Type 2 diabetes is related to obesity; most of the Type 2 diabetics are also obese. The compositions of the present invention are useful for treating both Type 1 and Type 2 diabetes. The compositions of the present invention are especially effective in treating diabetes associated with obesity. The term “diabetes associated with obesity” refers to diabetes caused by obesity or resulting from obesity. The compositions are especially effective for treating Type 2 diabetes. The compositions of the present invention are also useful for treating and/or preventing gestational diabetes mellitus.

Diabetes is characterized by a fasting plasma glucose level of greater than or equal to 126 mg/dl. A diabetic subject has a fasting plasma glucose level of greater than or equal to 126 mg/dl. Prediabetes is characterized by an impaired fasting plasma glucose (FPG) level of greater than or equal to 110 mg/dl and less than 126 mg/dl; or impaired glucose tolerance; or insulin resistance. A prediabetic subject is a subject with impaired fasting glucose (a fasting plasma glucose (FPG) level of greater than or equal to 110 mg/dl and less than 126 mg/dl); or impaired glucose tolerance (a 2 hour plasma glucose level of >140 mg/dl and <200 mg/dl); or insulin resistance, resulting in an increased risk of developing diabetes.

Treatment of diabetes mellitus refers to the administration of a combination of the present invention to treat a diabetic subject. One outcome of treatment may be decreasing the glucose level in a subject with elevated glucose levels. Another outcome of treatment may be decreasing insulin levels in a subject with elevated insulin levels. Another outcome of treatment may be decreasing plasma triglycerides in a subject with elevated plasma triglycerides. Another outcome of treatment is decreasing LDL cholesterol in a subject with high LDL cholesterol levels. Another outcome of treatment may be increasing HDL cholesterol in a subject with low HDL cholesterol levels. Another outcome of treatment is increasing insulin sensitivity. Another outcome of treatment may be enhancing glucose tolerance in a subject with glucose intolerance. Yet another outcome of treatment may be decreasing insulin resistance in a subject with increased insulin resistance or elevated levels of insulin. Prevention of diabetes mellitus, in particular diabetes associated with obesity, refers to the administration of a compound or combination of the present invention to prevent the onset of diabetes in a subject in need thereof. A subject in need of preventing diabetes is a prediabetic subject that is overweight or obese.

The term “hypertension” as used herein includes essential, or primary, hypertension wherein the cause is not known or where hypertension is due to greater than one cause, such as changes in both the heart and blood vessels; and secondary hypertension wherein the cause is known. Causes of secondary hypertension include, but are not limited to obesity; kidney disease; hormonal disorders; use of certain drugs, such as oral contraceptives, corticosteroids, cyclosporin, and the like. The term “hypertension” encompasses high blood pressure, in which both the systolic and diastolic pressure levels are elevated (>140 mmHg/>90 mmHg), and isolated systolic hypertension, in which only the systolic pressure is elevated to greater than or equal to 140 mm Hg, while the diastolic pressure is less than 90 mm Hg. Normal blood pressure may be defined as below 120 mm Hg (systolic pressure) over 80 mm Hg (diastolic pressure). A hypertensive subject is a subject with hypertension. A pre-hypertensive subject is a subject with a blood pressure that is between 120 mmHg over 80 mmHg and 139 mmHg over 89 mmHg. One outcome of treatment is decreasing blood pressure in a subject with high blood pressure. Treatment of hypertension refers to the administration of the combinations of the present invention to treat hypertension in a hypertensive subject. Prevention of hypertension refers to the administration of the combinations of the present invention to a pre-hypertensive subject to prevent the onset of hypertension or a hypertension related disorder.

Dyslipidemias or disorders of lipid metabolism, include various conditions characterized by abnormal concentrations of one or more lipids (i.e. cholesterol and triglycerides), and/or apolipoproteins (i.e., apolipoproteins A, B, C and E), and/or lipoproteins (i.e., the macromolecular complexes formed by the lipid and the apolipoprotein that allow lipids to circulate in blood, such as LDL, VLDL and IDL). Hyperlipidemia is associated with abnormally high levels of lipids, LDL and VLDL cholesterol, and/or triglycerides. Treatment of dyslipidemia refers to the administration of the combinations of the present invention to a dyslipidemic subject. Prevention of dyslipidemia refers to the administration of the combinations of the present invention to a pre-dyslipidemic subject. A pre-dyslipidemic subject is a subject with higher than normal lipid levels that is not yet dyslipidernic.

The term “Metabolic Syndrome” is defined in the Third Report of the National Cholesterol Education Program Expert Panel on Detection, Evaluation and Treatment of High Blood Cholesterol in Adults (ATP-III). E. S. Ford et al., JAMA, vol. 287 (3), Jan. 16, 2002, pp 356-359. Briefly, a person is defined as having Metabolic Syndrome if the person has three or more of the following disorders: abdominal obesity, hypertriglyceridemia, low HDL cholesterol, high blood pressure, and high fasting plasma glucose. The criteria for these are defined in ATP-III. Treatment of Metabolic Syndrome refers to the administration of the combinations of the present invention to a subject with Metabolic Syndrome. Prevention of Metabolic Syndrome refers to the administration of the combinations of the present invention to a subject with two of the disorders that define Metabolic Syndrome. A subject with two of the disorders that define Metabolic Syndrome is a subject that has developed two of the disorders that define Metabolic Syndrome, but has not yet developed three or more of the disorders that define Metabolic Syndrome.

Left ventricular hypertrophy (LVH) is identified based on left ventricular mass index (LVMI) and relative wall thickness (RWT). Left ventricular mass index is defined as left ventricular mass in grams divided by body surface area in meters². Relative wall thickness is defined as 2× posterior wall thickness/left ventricular end diastolic diameter. Normal LVMI values are typically 85 and normal RWT approximately 0.36. A male subject with LVH has a LVMI greater than 131 g/m²; a female subject with LVH has a LVMI greater than 100 g/m². A subject with an elevated LVMI value is a male subject with a LVMI between 85 g/m² and 131 g/m², or a female subject with a LVMI between 85 g/m² and 100 g/m². Treatment of cardiac hypertrophy, or left ventricular hypertrophy, refers to the administration of the combinations of the present invention to a subject with cardiac hypertrophy or left ventricular hypertrophy. Prevention of cardiac hypertrophy, or left ventricular hypertrophy, refers to the administration of the combinations of the present invention to decrease or maintain the LVMI in a subject with an elevated LVMI value or to prevent the increase of LVMI in a subject with a normal LVMI value.

One outcome of treatment of cardiac hypertrophy or left ventricular hypertrophy may be a decrease in ventricular mass. Another outcome of treatment of cardiac hypertrophy or left ventricular hypertrophy may be a decrease in the rate of increase of ventricular mass. Another outcome of treatment of cardiac hypertrophy or left ventricular hypertrophy may be a decrease in ventricular wall thickness. Another outcome of treatment of cardiac hypertrophy of left ventricular hypertrophy may be the decrease in the rate of increase in ventricular wall thickness. One outcome of treatment of diabetes while mitigating cardiac hypertrophy, or left ventricular hypertrophy, may be a decrease in ventricular mass. Another outcome of treatment of diabetes while mitigating cardiac hypertrophy or left ventricular hypertrophy may be a decrease in the rate of increase of ventricular mass. Another outcome of treatment of diabetes while mitigating cardiac hypertrophy or left ventricular hypertrophy may be a decrease in ventricular wall thickness. Another outcome of treatment of diabetes while mitigating cardiac hypertrophy of left ventricular hypertrophy may be the decrease in the rate of increase in ventricular wall thickness.

The term “obesity” as used herein is a condition in which there is an excess of body fat. The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asia-Pacific, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus (Type 2), diabetes associated with obesity, impaired glucose tolerance, impaired fasting glucose, insulin resistance syndrome, dyslipidemia, hypertension, hypertension associated with obesity, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility, lower back pain, and increased anesthetic risk. In particular, co-morbidities include: hypertension, hyperlipidemia, dyslipidemia, glucose intolerance, cardiovascular disease, sleep apnea, diabetes mellitus, and other obesity-related conditions.

Treatment of obesity and obesity-related disorders refers to the administration of the combinations of the present invention to reduce or maintain the body weight of an obese subject. One outcome of treatment may be reducing the body weight of an obese subject relative to that subject's body weight immediately before the administration of the compounds or combinations of the present invention. Another outcome of treatment may be preventing body weight regain of body weight previously lost as a result of diet, exercise, or pharmacotherapy and preventing weight gain from cessation of smoking. Another outcome of treatment may be decreasing the occurrence of and/or the severity of obesity-related diseases. Yet another outcome of treatment may be decreasing the risk of developing diabetes in an overweight or obese subject. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

Prevention of obesity and obesity-related disorders refers to the administration of the combinations of the present invention to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type 2 diabetes, polycystic ovary disease, cardiovascular diseases, osteoarthritis, dermatological disorders, hypertension, insulin resistance, hypercholesterolemia, hypertriglyceridemia, and cholelithiasis.

In particular, the compositions of the present invention are useful to the treatment of atherosclerosis. The term “atherosclerosis” as used herein encompasses vascular diseases and conditions that are recognized and understood by physicians practicing in the relevant fields of medicine. Atherosclerotic cardiovascular disease, coronary heart disease (also known as coronary artery disease or ischemic heart disease), cerebrovascular disease and peripheral vessel disease are all clinical manifestations of atherosclerosis and are therefore encompassed by the terms “atherosclerosis” and “atherosclerotic disease.” The composition comprised of a therapeutically or prophylactically effective amount of a DPP-IV inhibitor in combination with a therapeutically or prophylactically effective amount of a cannabinoid CB₁ receptor antagonist/inverse agonist may be administered to prevent or reduce the risk of occurrence, or recurrence where the potential exists, of a coronary heart disease event, a cerebrovascular event, or intermittent claudication. Coronary heart disease events are intended to include CHD death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events, and intermittent claudication. It is intended that persons who have previously experienced one or more non-fatal atherosclerotic disease events are those for whom the potential for recurrence of such an event exists.

The terms “administration of” and or “administering a” composition should be understood to mean providing a composition of the invention to a subject in need of treatment. The instant pharmaceutical compositions include administration of a single pharmaceutical dosage formulation which contains a DPP-IV inhibitor and a cannabinoid CB₁ receptor antagonist/inverse agonist, as well as administration of each active agent in its own separate pharmaceutical dosage formulation. Where separate dosage formulations are used, the individual components of the composition can be administered at essentially the same time, i.e., concurrently, or at separately staggered times, i.e. sequentially prior to or subsequent to the administration of the other component of the composition. The instant pharmaceutical composition is therefore to be understood to include all such regimes of simultaneous or alternating treatment, and the terms “administration” and “administering” are to be interpreted accordingly. Administration in these various ways are suitable for the present compositions as long as the beneficial pharmaceutical effect of the combination of the anti-obesity agent and the anti-diabetic agent is realized by the patient at substantially the same time. Such beneficial effect is preferably achieved when the target blood level concentrations of each active drug are maintained at substantially the same time. It is preferred that the combination of the DPP-IV inhibitor and the cannabinoid CB₁ receptor antagonist/inverse agonist be co-administered concurrently on a once-a-day dosing schedule; however, varying dosing schedules, such as the anti-obesity agent once a day and the anti-diabetic agent once, twice or more times per day, is also encompassed herein. A single oral dosage formulation comprised of both agents in the combination is preferred. A single dosage formulation will provide convenience for the patient, which is an important consideration especially for patients with diabetes, Metabolic Syndrome, or obese patients who may be in need of multiple medications.

The term “subject”, as used herein refers to a mammal, preferably a human, who has been the object of treatment, observation or experiment. In one embodiment the term “mammal” is a “human” said human being either male or female. The instant combinations are also useful for treating or preventing obesity and obesity-related disorders in cats and dogs. As such, the term “mammal” includes companion animals such as cats and dogs.

The term “subject in need thereof” refers to a subject who is in need of treatment or prophylaxis as determined by a researcher, veterinarian, medical doctor or other clinician. In one embodiment, a subject in need thereof is a mammal. In another embodiment, a subject in need thereof is an obese subject. In another embodiment, a subject in need thereof is an obese subject with diabetes. In another embodiment, a subject in need thereof is an obese subject at risk of developing diabetes. In another embodiment, a subject in need thereof is a diabetic subject. In another embodiment, a subject in need thereof is an obese diabetic subject. In another embodiment, a subject in need thereof is a diabetic subject at risk of developing obesity. In another embodiment, a subject in need thereof is an obese subject with Metabolic Syndrome. In another embodiment, a subject in need thereof is an obese subject at risk of developing Metabolic Syndrome. In another embodiment, a subject in need thereof is a diabetic subject with Metabolic Syndrome. In another embodiment, a subject in need thereof is a diabetic subject at risk of developing Metabolic Syndrome. In another embodiment, a subject in need thereof is an obese diabetic subject with Metabolic Syndrome. In another embodiment, a subject in need thereof is an obese subject at risk of developing Metabolic Syndrome. In another embodiment, a subject in need thereof is a diabetic subject at risk of developing Metabolic Syndrome. In another embodiment, a subject in need thereof is an obese diabetic subject at risk of developing Metabolic Syndrome.

In another embodiment, a subject in need thereof is an obese subject with cardiac hypertrophy, or left ventricular hypertrophy. In another embodiment, a subject in need thereof is a diabetic subject with cardiac hypertrophy, or left ventricular hypertrophy. In another embodiment, a subject in need thereof is an obese diabetic subject with cardiac hypertrophy, or left ventricular hypertrophy. In another embodiment, a subject in need thereof is an obese subject at risk of developing cardiac hypertrophy, or left ventricular hypertrophy. In another embodiment, a subject in need thereof is a diabetic subject at risk of developing cardiac hypertrophy, or left ventricular hypertrophy. In another embodiment, a subject in need thereof is an obese diabetic subject at risk of developing cardiac hypertrophy, or left ventricular hypertrophy. In another embodiment, a subject in need thereof is an obese diabetic subject with cardiac hypertrophy, or left ventricular hypertrophy, undergoing PPARγ agonist treatment. In another embodiment, a subject in need thereof is an obese diabetic subject undergoing PPARγ agonist treatment and at risk of developing cardiac hypertrophy, or left ventricular hypertrophy.

The administration of the composition of the present invention in order to practice the present methods of therapy is carried out by administering a therapeutically or prophylactically effective amount of the compounds in the composition to a subject in need of such treatment or prophylaxis. The need for a prophylactic administration according to the methods of the present invention is determined via the use of well known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgment.

The term “therapeutically effective amount” as used herein means the amount of the active compounds in the composition that will elicit the biological or medical response in a tissue, system, subject, or human that is being sought by the researcher, veterinarian, medical doctor or other clinician, which includes alleviation of the symptoms of the disorder being treated. The novel methods of treatment of this invention are for disorders known to those skilled in the art.

The term “prophylactically effective amount” as used herein means the amount of the active compounds in the composition that will elicit the biological or medical response in a tissue, system, subject, or human that is being sought by the researcher, veterinarian, medical doctor or other clinician, to prevent the onset of diabetes, diabetes associated with obesity, a diabetes associated disorder, obesity or an obesity-related disorder in a subject at risk of developing the disorder.

The magnitude of prophylactic or therapeutic dose of the active ingredients of the composition may vary with the nature of the severity of the condition to be treated and with the particular compound in the composition and its route of administration. It will also vary according to the age, weight and response of the individual patient. In general, the daily dose range of each compound in the combination lies within the range of from about 0.0001 mg/kg to about 100 mg/kg, preferably from about 0.001 mg/kg to about 50 mg/kg body weight of a subject in single or divided doses. On the other hand, it may be necessary to use dosages outside these limits in some cases.

In the case where an oral composition is employed, a suitable dosage range is, e.g. from about 0.001 mg/kg to about 100 mg/kg of each compound in the composition per day, preferably from about 0.01 mg to about 2000 mg per day. For oral administration, the compositions are preferably provided in the form of tablets containing from 0.01 mg to 1,000 mg, e.g. 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850, 1,000 and 2,000 milligrams of each active ingredient for the symptomatic adjustment of the dosage to the subject to be treated. This dosage regimen may be adjusted to provide the optimal therapeutic response.

The anti-diabetic DPP-IV inhibitors in the combinations of the present invention are administered at a daily dosage of from about 0.1 mg to about 100 mg per kilogram of animal body weight, preferably given as a single daily dose or in divided doses two to six times a day, or in sustained release form. For most large mammals, the total daily dosage is from about 1.0 mg to about 1000 mg, preferably from about 10 mg to about 200 mg. In the case of a 70 kg adult human, the total daily dose will generally be from about 7 mg to about 350 mg. This dosage regimen may be adjusted to provide the optimal therapeutic response.

In one embodiment the substituted amide cannabinoid CB₁ receptor antagonist/inverse agonists disclosed in international patent publication WO 2003/077847 are administered at a total daily dosage of from about 1.0 mg to about 1000 mg, preferably from about 1 mg to about 50 mg. In the case of a 70 kg adult human, the total daily dose will generally be from about 1 mg to about 35 mg. This dosage regimen may be adjusted to provide the optimal therapeutic response.

The effective dosage of each of the active ingredients employed in the composition may vary depending on the particular compound employed, the mode of administration, the condition being treated and the severity of the condition being treated. Thus, the dosage regimen utilizing the compositions of the present invention is selected in accordance with a variety of factors including type, species, age, general health, body weight, diet, sex and medical condition of the subject; the severity of the condition to be treated; the renal and hepatic function of the patient; the drug combination; and the particular compounds employed and their routes of administration. A physician, clinician or veterinarian of ordinary skill can readily determine and prescribe the effective amount of the drug required to prevent, counter or arrest the progress of the condition.

The weight ratio of the agents in the combinations of the present invention may be varied and will depend upon the effective dose of each ingredient. Generally, an effective dose of each will be used.

Another aspect of the present invention provides pharmaceutical compositions comprising a pharmaceutical carrier and a therapeutically or prophylactically effective amount of each compound in the composition of the present invention. The term “composition”, as in pharmaceutical composition, is intended to encompass a product comprising the active ingredient(s), and the inert ingredient(s), such as pharmaceutically acceptable excipients, that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing an anti-obesity agent and an anti-diabetic agent, and pharmaceutically acceptable excipients.

Any suitable route of administration may be employed for providing a subject, especially a human, with an effective dosage of a composition of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a combination of a DPP-IV inhibitor and a cannabinoid CB₁ receptor antagonist/inverse agonist, as active ingredients, or a pharmaceutically acceptable salt or ester thereof, and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. In particular, the term “pharmaceutically acceptable salts” refers to salts prepared from pharmaceutically acceptable non-toxic bases or acids including inorganic bases or acids and organic bases or acids.

The compositions include compounds suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. These compositions may be conveniently presented in unit dosage form and prepared by any of the methods well-known in the art of pharmacy.

For administration by inhalation, the compositions of the present invention are conveniently delivered in the form of an aerosol spray presentation from pressurized packs or nebulizers. The compositions may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of the instant composition in suitable propellants, such as fluorocarbons or hydrocarbons and dry powder inhalation (DPI) aerosol, which may be formulated as a dry powder of the composition with or without additional excipients.

Suitable topical formulations of the compositions of the present invention include transdermal devices, aerosols, creams, solutions, ointments, gels, lotions, dusting powders, and the like. The topical pharmaceutical compositions containing the compositions of the present invention ordinarily include about 0.005% to 5% by weight of the active compounds in admixture with a pharmaceutically acceptable vehicle. Transdermal skin patches useful for administering the compositions of the present invention include those well known to those of ordinary skill in that art. To be administered in the form of a transdermal delivery system, the dosage administration will, of course be continuous rather than intermittent throughout the dosage regimen.

The compositions of the present invention can also be administered in the form of liposome delivery systems, such as small unilamellar vesicles, large unilamellar vesicles and multilamellar vesicles. Liposomes can be formed from a variety of phospholipids, such as cholesterol, sterylamine or phosphatidylcholines.

Compositions of the present invention may also be delivered by the use of monoclonal antibodies as individual carriers to which the compound molecules are coupled. The compounds in these compositions may also be coupled with soluble polymers as targetable drug carriers. Such polymers can include polyvinylpyrrolidone, pyran copolymer, polyhydroxypropyl-methacrylamide phenol, polyhydroxyethylasparamidepheon, or polyethyleneoxidepolylysine substituted with palmitoyl residues. Furthermore, the compositions of the present invention may be coupled to a class of biodegradable polymers useful in achieving controlled release of a drug, for example, polylactic acid, polyepsilon caprolactone, polyhydroxybutyric acid, polyorthoesters, polyacetals, polydihydropyrans, polycyanoacrylates and cross-linked or amphipathic block copolymers of hydrogels.

Compositions of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

In practical use, each compound in the compositions of the present invention can be combined as the active ingredients in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules, pellet, powder and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the composition may also be administered by controlled release means and/or delivery devices such as those described in U.S. Pat. Nos. 3,845,770; 3,916,899; 3,536,809; 3,598,123; 3,630,200; and 4,008,719.

Pharmaceutical compositions of the present invention suitable for oral administration may be presented as discrete units such as capsules (including timed release and sustained release formulations), pills, cachets, powders, granules or tablets each containing a predetermined amount of the active ingredients, as a powder or granules or as a solution or a suspension in an aqueous liquid, a non-aqueous liquid, an oil-in-water emulsion or a water-in-oil liquid emulsion, including elixirs, tinctures, solutions, suspensions, syrups and emulsions. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

For example, for oral administration in the form of a tablet, capsule, pellet, or powder, the active ingredient can be combined with an oral, non-toxic, pharmaceutically acceptable inert carrier such as lactose, starch, sucrose, glucose, methyl cellulose, magnesium stearate, mannitol, sorbitol, croscarmellose sodium and the like; for oral administration in liquid form, e.g., elixirs, syrups, slurries, emulsions, suspensions, solutions, and effervescent compositions, the oral drug components can be combined with any oral, non-toxic, pharmaceutically acceptable inert carrier such as ethanol, glycerol, water, oils and the like. Moreover, when desired or necessary, suitable binders, lubricants, disintegrating agents, buffers, coatings, and coloring agents can also be incorporated. Suitable binders can include starch, gelatin, natural sugars such a glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. When a dosage unit form is a capsule, it may contain, in addition to materials of the above type, a liquid carrier such as a fatty oil.

Desirably, each tablet contains from 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active ingredient in the composition of the present invention for the symptomatic adjustment of the dosage to the subject to be treated; and each cachet or capsule contains from about 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active ingredient in the composition of the present invention for the symptomatic adjustment of the dosage to the subject to be treated.

Exemplifying the invention is a pharmaceutical composition comprising a DPP-IV inhibitor and a CB₁ cannabinoid receptor antagonist/inverse agonist described above and a pharmaceutically acceptable carrier.

Also exemplifying the invention is a pharmaceutical composition made by combining any of the DPP-IV inhibitors and cannabinoid CB₁ receptor antagonist/inverse agonists described above and a pharmaceutically acceptable carrier. An illustration of the invention is a process for making a pharmaceutical composition comprising combining any of the anti-obesity agents and anti-diabetic agents described above and a pharmaceutically acceptable carrier.

The dose may be administered in a single daily dose or the total daily dosage may be administered in divided doses of two to six times daily. Furthermore, based on the properties of the individual compound selected for administration, the dose may be administered less frequently, e.g., weekly, twice weekly, monthly, etc. The unit dosage will, of course, be correspondingly larger for the less frequent administration.

When administered via intranasal routes, transdermal routes, by rectal or vaginal suppositories, or through a continual intravenous solution, the dosage administration will, of course, be continuous rather than intermittent throughout the dosage regimen.

The following is an examples of a representative pharmaceutical dosage form for the compositions of the present invention:

| Tablet | 150 mg/tablet | |
|---|---|---|
| Compound of formula III | 10 | mg |
| MK-0431* | 64.25 | mg |
| Microcrystalline Cellulose | 53.19 | mg |
| Hydroxypropylcellulose | 9 | mg |
| Croscarmellose Sodium | 9 | mg |
| Magnesium Stearate | 4.5 | mg |
| Butylated hydroxyanisole (BHA) | 0.06 | mg |

64.25 mg of the dihydrogenphosphate salt is equivalent to 50 mg of the free base

### Method of Manufacture:

The steps involved in the direct compression method comprise:
(1) blending Compound of formula III, MK-0431, and croscarmellose sodium in a V-blender or other suitable blender for a period of about 5 to 30 min;
(2) adding hydroxypropyl cellulose and microcrystalline cellulose to improve compaction properties;
(3) lubricating with magnesium stearate for about 1 to 15 min;
(4) compressing the lubricated blend into a desired tablet image; and, if desired,
(5) film-coating.

An antioxidant, such as BHA or BHT, can be added by either layering it onto one of the excipients prior to blending with Compound of formula III and MK-0431 and the other excipients or by layering it onto Compound of formula III and MK-0431 during the bulk drug synthesis process. The tablets are optionally coated with 6.00 mg of a standard HPC/HPMC/TiO₂ film-coat formula (Opadry I®) to provide a 156-mg coated tablet.

It will be understood that the scope of compositions of the compounds of this invention with other agents useful for treating or preventing obesity and obesity-related conditions includes in principle any combination with any pharmaceutical composition useful for treating obesity and obesity-related disorders.

In order to illustrate the invention, the following examples are included. These examples do not limit the invention. They are only meant to suggest a method of reducing the invention to practice. Those skilled in the art may find other methods of practicing the invention which are readily apparent to them. However, those methods are also deemed to be within the scope of this invention.

The substituted amide cannabinoid CB₁ receptor antagonist/inverse agonists from international patent publication WO 2003/077847 (published 24 Sep. 2003) were prepared following the synthetic methods disclosed therein and exemplified by the synthesis of N-[(1S,2S)-3-(4-chlorophenyl)-2-(3-cyanophenyl)-1-methylpropyl]-2-methyl-{[5-(trifluoromethyl)pyridin-2-yl]oxy}propanamide of structural formula III below:

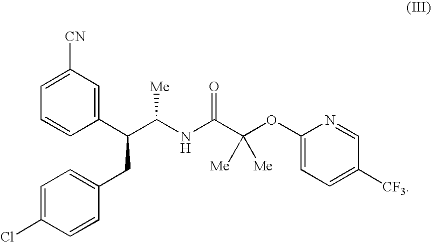

### Preparation of Intermediate 2-methyl-2-{[5-(trifluoromethyl)pyridin-2-yl]oxy}propanoic acid (1)

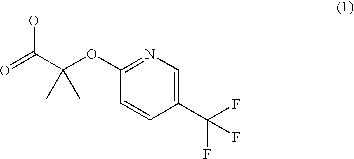

Step 1: Ethyl 2-methyl-2-{[5-(trifluoromethyl)pyridin-2-yl]oxy}propanoate

A mixture of 2-hydroxy-5-trifluoromethylpyridine (5.73 g, 39 mmol), ethyl 2-bromoisobutyrate (5.7 mL, 39 mmol) and cesium carbonate (25 g, 77 mmol) in 50 mL acetonitrile was heated at 5° C. overnight. The volatile materials were removed by concentrating on a rotary evaporator, and the residue was partitioned between water (100 mL) and EtOAc (100 mL). The organic layer was separated and the aqueous layer extracted with EtOAc (2×100 mL). The combined organic extracts were dried over anhydrous sodium sulfate, filtered and concentrated to dryness, and the residue was purified by flash column chromatography on silica gel eluted with 5% EtOAc in hexane to give the title compound. ¹H NR (500 MHz, CD₃OD): δ 7.99 (d, 1H), 7.67 (dd, 1H), 6.68 (d, 1H), 4.13 (q, 2H), 1.64 (s, 6H), 1.14 (t, 3H). LC-MS: m/e 244 (M+H)⁺ (3.41 min).

Step 2: 2-Methyl-2-{[5-(trifluoromethyl)pyridin-2-yl]oxy}propanoic acid (1)

A mixture of ethyl 2-methyl-2-{[5-(trifluoromethyl)pyridin-2-yl]oxy}propanoate and sodium hydroxide (0.85 g, 21 mmol) in 15 mL acetonitrile and 15 mL water was heated at 50° C. overnight. The volatile materials were removed by concentrating on a rotary evaporator, and the residue was partitioned between 2 M hydrochloric acid (100 mL) and ether (100 mL). The organic layer was separated and washed with water (2×50 mL), dried over anhydrous MgSO₄, filtered and concentrated to dryness to give the title compound. ¹H NMR (500 MHz, CD₃OD): δ 8.38 (br s, 1H), 7.93 (dd, 1H), 7.13 (d, 1H), 1.70 (s, 6H). LC-MS: m/e 250 (M+H)⁺ (2.6 min).

Step A: Preparation of 2-methyl-2-{[5-(trifluoromethyl)pyridin-2-yl]oxy}-propanamide (2)

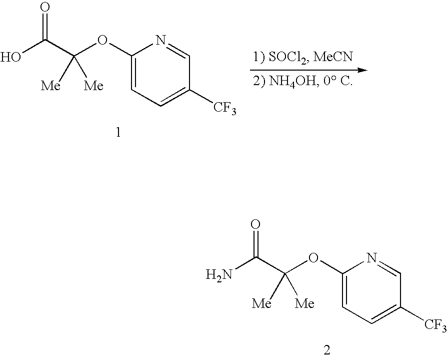

In a 12 L 3-neck separatory funnel equipped with overhead stirrer, nitrogen inlet and thermocouple, a solution of 2-methyl-2-{[5-(trifluoromethyl)pyridin-2-yl]oxy}propanoic acid (1) (772 g) in MeCN (6.5 L) was prepared. Thionyl chloride (316 mL) was added over 30 min. The resulting solution was stirred at room temperature for 2 h. A separate 22 L 3-necked round bottom flask equipped with overhead stirrer, nitrogen inlet and thermocouple, was charged with 30% aqueous NH₄OH (5 L) and cooled to −20° C. The acid chloride solution from the separatory funnel was added to the solution of NH₄OH at such a rate that the internal reaction temperature was kept at −15 to −20° C. over 2 h. Once the addition was complete, the resulting slurry was warmed to room temperature and stirred for an additional 1 h. The reaction mixture was transferred to a 50-L extractor containing toluene (15 L) and water (15 L), and the layers were separated. The organic layer was washed with saturated aqueous NaHCO₃ (5 L), and then with water (5 L). The organic layer was transferred to a 12-L four-neck round bottom flask, and concentrated under vacuum at 50° C. to about 2 L volume. Near the end of the concentration, the solid began to precipitate, and the batch was heated to 78° C. to dissolve all of the solids. Heptane (5 L) was added and the batch was allowed to slowly cool, affording a crystalline solid. The slurry was filtered, and the filter cake was washed with n-heptane (1 L). The resulting solid was dried under a stream of nitrogen to afford the title compound.

Step B: Preparation of 3-[1-(4-chlorobenzyl)-2-oxopropyl]benzonitrile (4)

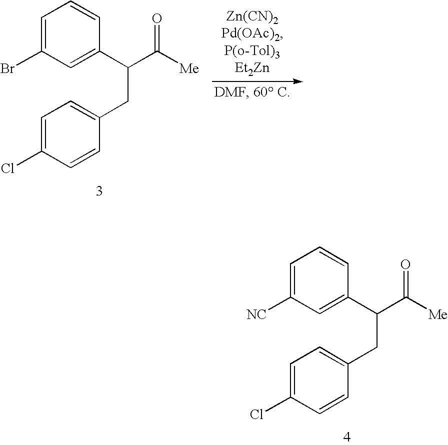

### Catalyst Preparation:

A 4-neck 12 L round bottom flask equipped with thermocouple, overhead stirrer, rubber septum, N₂ inlet, and gas adapter connected to a bubbler was charged with palladium acetate (12.8 g), tri-o-tolylphosphine (69.9 g), and N,N-dimethylformamide (DMF) (2.8 L). N₂ was bubbled through the solution for 20 min at ambient temperature. The flask was then heated to 56° C. on a heating mantle, and the mixture was stirred at 60° C. for 20 min. A solution of diethylzinc in toluene (1.1M, 78.0 mL) was added via syringe. The resulting suspension was stirred at 56° C. for 45 min.

### Cyanation Reaction:

A 4-neck 12 L flask equipped with a mechanical stirrer, thermocouple, nitrogen inlet, and gas adapter connected to a bubbler was charged with 3-(3-bromophenyl)-4-(4-chlorophenyl)butan-2-one (3), zinc cyanide (201 g), and N,N-dimethylformamide (4.0 L). Nitrogen gas was bubbled through the suspension for 30 min at room temperature and for 1 h at 56° C. using a heating mantle. The bromoketone/Zn(CN)₂ slurry (at 56° C.) was added to the catalyst solution (at 56° C.). After the transfer was complete, the reaction mixture was stirred at 56° C. for 4.5 h under N₂. The resulting suspension was cooled in an ice bath, and 30% aqueous ammonium hydroxide (971 mL) was added over 5 min, keeping the temperature below 30° C. The suspension was warmed to room temperature, stirred for 60 min, and then filtered through a pad of SOLKA FLOC eluting with toluene (5 L). The filtrate was added into an extractor containing 20% aqueous ammonium hydroxide (6.9 L) and 5 L of toluene. The biphasic mixture was stirred at room temperature for 15 min and then separated. The organic layer was washed with 7 L of brine (1:1 saturated NaCl:water), then 7 L of water. The organic phase was transferred to a 12-L 4-neck flask equipped with an overhead stirrer, thermocouple, mechanical stirrer, and connected to a batch concentrator. The batch was concentrated under vacuum at 15-38° C. to a volume of 1.5 L, and then heptane (850 mL) was added. A sample was taken at this point, and crystallized in a vial. This seed sample was recharged back to the flask which created a seed bed for the crystallization. Once a seed bed formed (about 30 min), 6.5 L of heptane was added over 40 min, and the batch was cooled to 0° C. The batch was filtered, and the filter cake was washed with heptane (2 L). The resulting solid was dried under a stream of nitrogen to provide the title compound.

Step C: 3-(4-chlorophenyl)-2-(3-cyanophenyl-1-methylprop-1-en-1-yl4-methylbenzenesulfonate (5)

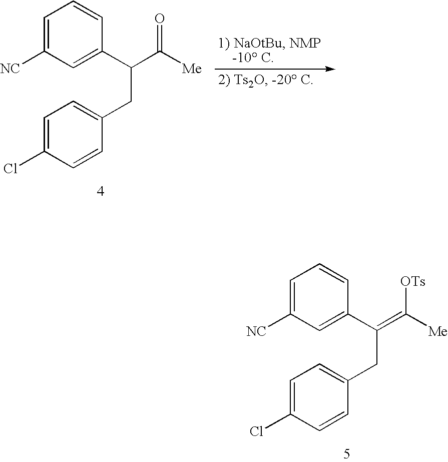

A 4-neck, 12 L flask equipped with a mechanical stirrer, thermocouple, and nitrogen inlet was charged N,N-dimethylacetamide (7.2 L), followed by 3 [1-(4-chlorobenzyl)-2-oxopropyl]benzonitrile (4, 744 g), then bubbled with nitrogen through the reaction mixture for 30 min at room temperature. The mixture was cooled to −10° C., and NaOtBu (265 g) was added as a solid in one portion with stirring (tmax=−2° C.). The solution was allowed to stir with cooling until the exotherm stopped and the temperature began to drop (about 2 min). The cooling bath was removed, and the reaction was warmed to room temperature, then stirred for 1 h. The mixture was cooled to −20° C. and p-toluenesulfonic anhydride (Ts₂O, 893 g) was added as a solid in two portions with stirring, keeping the temperature below −5° C. (tmax=−8° C.). The mixture was allowed to cool back to −10° C. and stirred for 1 h. The reaction was quenched with 1 M NaHCO₃ (1.9 L), and transferred to a 50 L extractor containing 15 L IPAc and 13 L water. The layers were separated and the organic layer was washed twice with 7.5 L water. The organic layer was concentrated under slight vacuum (25 in Hg) at 55° C. to about 2 L. Upon reaching the 2 L volume, the batch began to crystallize, so the vacuum was turned off and the flask was heated to 73° C. to produce a homogeneous solution. Heptane (6.6 L) was added while the mixture was allowed to slowly cool to room temperature. The resulting slurry was aged for 1 h at room temperature, then filtered. The filter cake was washed with 3 L heptane and dried under a stream of nitrogen to yield the title compound.

Step D: N-[(1Z)-3-(4-chlorophenyl)-2-(3-cyanophenyl)-1-methylprop-1-en-1-yl]-2-methyl-2-{[5-(trifluoromethyl)pyridin-2-yl]oxy}propanamide (6)

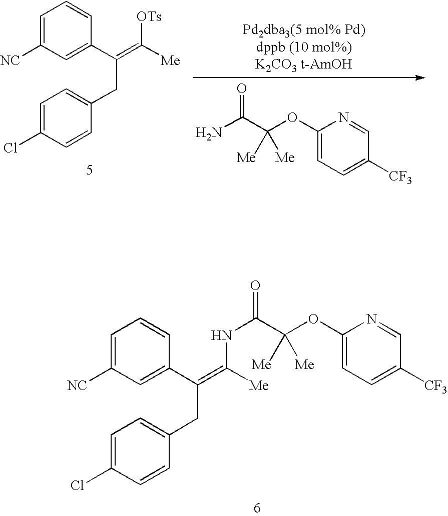

A 3-neck 3 L round bottom flask was charged with tert-amyl alcohol (2.4 L). Nitrogen gas was bubbled through the solution for 2 h. A 3-neck 5 L round bottom flask fitted with a mechanical stirrer, reflux condenser, and a nitrogen/vacuum adapter on top of the reflux condenser was charged with Pd₂dba₃ (27.5 g), 1,4-bis(diphenylphosphino)butane (51.2 g), 2-methyl-2-{[5-(trifluoromethyl)pyridine-2-yl]oxy}propanamide (2, 313 g), 3-(4-chlorophenyl)-2-(3-cyanophenyl)-1-methylprop-1-en-1-yl4-methylbenzenesulfonate (5, 526 g), and potassium carbonate (332 g). The flask was sealed, evacuated, and backfilled with nitrogen. Tert-amyl alcohol (2.4 L) was added to the reaction flask followed by heating to 100° C. and stirring at 100° C. for 18 h. The resulting suspension was cooled to 25° C. and transferred into a 4-necked 22 L round bottom flask equipped with a mechanical stirrer. The batch was diluted with 7.2 L of MTBE, then DARCO KB-B® (250 g) was charged to the mixture. The resulting mixture was stirred for 2 h at RT, then filtered over a pad of SOLKA FLOC. The filter cake was washed with 7 L of MTBE. The batch was vacuum transferred to a 4-necked 12 L round bottom flask equipped with an overhead stirrer and thermocouple. The batch was concentrated at 10-20° C. to remove all the MTBE and then at 30-40° C. to reduce the volume of the remaining t-amyl alcohol to ˜1.5 L. Heptane (5 L) was added over ˜30 minutes and the batch was cooled to 20° C. The filter cake was washed with 2 L of heptane-MTBE (10:1) and dried under a stream of nitrogen to provide the title compound.

Step E: 3-{(1Z)-1-(4-chlorobenzyl)-2-[(2-methyl-2-{[5-(trifluoromethyl)pyridin-2-yl]oxy}propanoyl)amino]-prop-1-en-1-yl}benzamide (7)

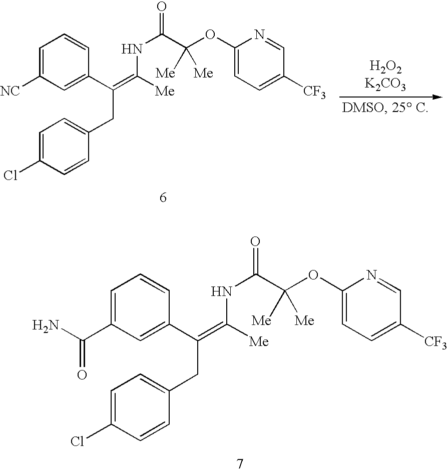

To a 5 L, 3-necked round bottom flask equipped with overhead stirrer, thermocouple, and nitrogen inlet was added 524 g of the cyano enamide 6 and 112 g K₂CO₃. DMSO (2.7 L) was charged and the vessel was submerged in a RT water bath. Hydrogen peroxide solution (165 mL of a 30% aqueous solution) was slowly added to the reactor such that the temperature never rose above 25° C. After the addition was complete, the reaction was aged for 1 h. The batch was diluted with 1 L of isopropyl acetate and filtered over a bed of SOLKA FLOC. The bed was washed with 4.5 L of isopropyl acetate and the resulting solution was transferred to a 50 L extractor containing 5.5 L of water. The layers were separated and the organic layer was washed twice with 3.1 L of water, concentrated to 5 L, and solvent switched to 5 L toluene at about 60° C. Upon completion of the solvent switch, 500 mL of heptane was added and the mixture was cooled to 20° C. The batch was aged for 30 min at 20° C., then filtered and washed with 1 L of toluene. The resulting solid was dried overnight under a stream of nitrogen to afford the title compound.

Step F: 3-{(1S,2S)-1-(4-chlorobenzyl)-2-[(2-methyl-2-{[5-(trifluoromethylpyridine-2-yl]oxy}propanoyl)amino]-propyl}benzamide (8)

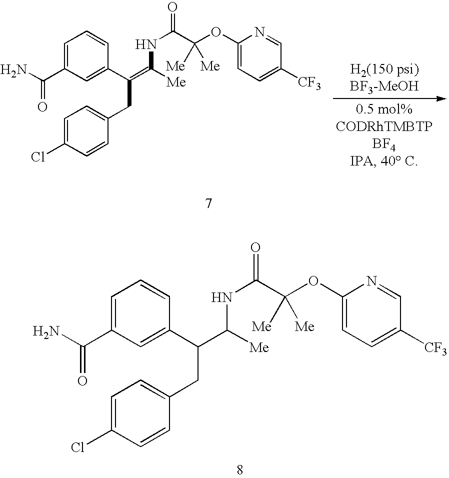

### Catalyst Preparation:

In a N₂-filled glove box, 2.83 g (−)-TMBTP was added to a 0.5 L bottle containing a stir bar. (COD)₂Rh BF₄ (1.85 g) was added to the same bottle and then methanol (360 mL) was added. The resulting solution was aged with stirring for 1 h. BF₃—MeOH (41.2 g, 12 wt % in MeOH, 4.94 g BF₃) was added to the catalyst solution, and the resulting mixture was added to a 1-L stainless steel bomb. 50 mL of MeOH was used to rinse the mixture into the bomb. Isopropanol (200 mL) was charged to the rinse chamber of the bomb, and then each chamber of the bomb was sealed before removing it from the glove box.

### Hydrogenation:

Compound 7 was charged to a 5 L bottle, and isopropanol (3.3 L) was added to the bottle to create a slurry. The resulting slurry was transferred by vacuum through a polyethylene line into a 2 gallon stainless steel autoclave. The 5-L bottle was rinsed with 1 L of isopropanol and the rinse was also transferred into the 2 gallon autoclave. The autoclave was degassed five times with N₂, and then placed under partial vacuum. The catalyst bomb was connected to the autoclave via flexible polyethylene tubing (flushed with N₂) and the catalyst solution was drawn into the autoclave followed by the isopropanol wash from the rinse chamber. The autoclave was sealed, degassed with N₂ purges three times and pressurized up to 150 psi. The stirrer was initiated, and the temperature was raised to 40° C. The reaction was aged at 150 psi, 40° C. for 18 h. The temperature was dropped to room temperature, and the resulting solution was transferred to a polyethylene jug.

Step G: N-[1S,2S]-3-(4-chlorophenyl)-2-(3-cyanophenyl)-1-methylpropyl]-2-methyl-2-{[5-(trifluoromethyl)pyridin-2-yl]oxy}propanamide (9)

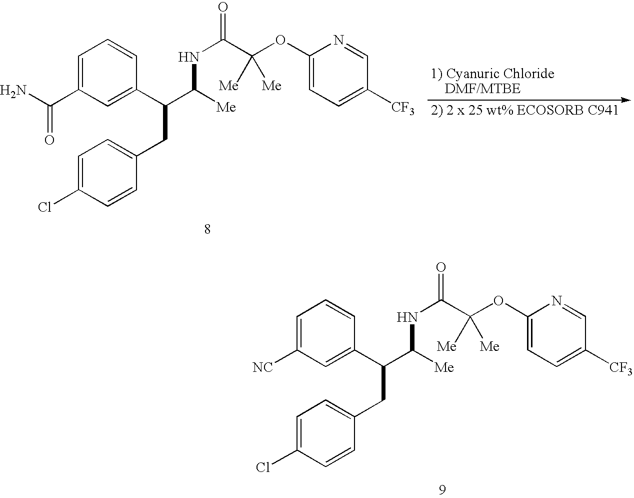

The crude hydrogenation solution from Step F was solvent switched from 4 L isopropanol to ˜1 L DMF (40° C., 30 mm Hg). The resulting solution of 470 g of intermediate 8 in DMF was transferred to a 12 L 4-necked round bottom flask equipped with mechanical stirrer, thermocouple, and 2 L addition funnel. Cyanuric chloride (103 g) was slurried in 2 L of MTBE and the resulting slurry was charged to the reaction via the 2 L addition funnel over about 10 min. The reaction mixture was aged with stirring for 1 h. The batch was cooled to 10° C. and diluted with 3 L of MTBE. 2 L of water and 2 L of saturated NaHCO₃ solution were added to the reaction while keeping the temperature below 20° C. The resulting slurry was transferred to a 50 L extractor containing 3 L of MTBE, 3 L of water, and 3 L of saturated aqueous NaHCO₃. An additional 12 L of water was added to the batch and the layers were allowed to settle. The organic layer was washed twice with 3 L of water. The organic layer was azeotroped at 35° C., 17 in Hg to bring the KF to 219 (spec. at 500) while maintaining a volume of about 11 L. The batch was then treated with 320 g of ECOSORB C941. The batch was aged for 4 h at 50° C., then filtered over a pad of SOLKA FLOC and washed with 6 L of MTBE. The resulting filtrate was recharged to a 22 L vessel, concentrated to 11 L volume, and retreated with 116 g of ECOSORB C941. This slurry was filtered over a bed of SOLKA FLOC, and washed with 6 L MTBE. The resulting colorless MTBE layer was transferred through a 1 micron inline filter into a 12 L, 4 neck round bottom flask equipped with overhead stirrer and thermocouple, and concentrated to about 2 L volume at 17 in Hg, 35° C. The batch was cooled to room temperature, and a sample was removed to create a seed bed. Once the sample crystallized, it was returned to the flask, and the batch was aged for 30 minutes, creating a large seed bed. The isolated solid was dried over a stream of nitrogen to afford the title compound as a hemisolvate.

The DPP-IV inhibitors including the compound of structural formula IV for use in the compositions of the present invention were prepared as described in U.S. Pat. No. 6,699,871, the contents of which are incorporated herein by reference in their entirety. The dihydrogenphosphate salt of structural formula V and its crystalline monohydrate form were prepared as described below.

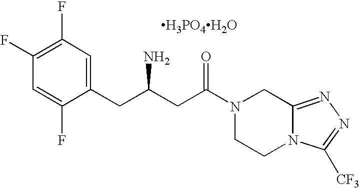

### (2R)-4-oxo-4-[3-(trifluoromethyl)-5,6-dihydro[1,2,4]triazolo-4,3-α]pyrazin-7(8H)-yl]-1-(2,4,5-trifluorophenyl)butan-2-amine dihydrogenphosphate monohydrate (MK-0431)

### Preparation of 3-(trifluoromethyl)-5,6,7,8-tetrahydro[1,2,4]triazolo[4,3-α]pyrazine hydrochloride (1-4)

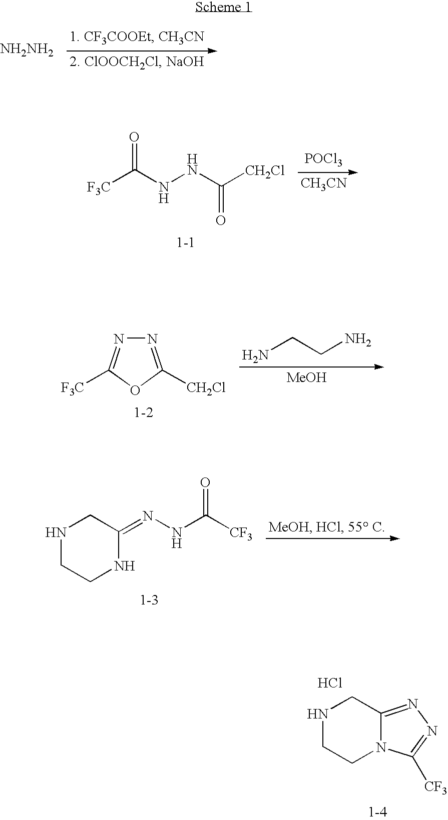

Step A: Preparation of bishydrazide (1-1)

Hydrazine (20.1 g, 35 wt % in water, 0.22 mol) was mixed with 310 mL of acetonitrile. 31.5 g of ethyl trifluoroacetate (0.22 mol) was added over 60 min. The internal temperature was increased to 25° C. from 14° C. The resulting solution was aged at 22-25° C. for 60 min. The solution was cooled to 7° C. 17.9 g of 50 wt % aqueous NaOH (0.22 mol) and 25.3 g of chloroacetyl chloride (0.22 mol) were added simultaneously over 130 min at a temperature below 16° C. When the reaction was complete, the mixture was vacuum distilled to remove water and ethanol at 27˜30° C. and under 26˜27 in Hg vacuum. During the distillation, 720 mL of acetonitrile was added slowly to maintain constant volume (approximately 500 mL). The slurry was filtered to remove sodium chloride. The cake was rinsed with about 100 mL of acetonitrile. Removal of the solvent afforded bis-hydrazide 1-1.

¹H-NMR (400 MHz, DMSO-d₆): δ 4.2 (s, 2H), 10.7 (s, 1H), and 11.6 (s, 1H) ppm.

¹³C-NMR (100 MHz, DMSO-d₆): δ 41.0, 116.1 (q, J=362 Hz), 155.8 (q, J=50 Hz), and 165.4 ppm.

Step B: Preparation of 5-(trifluoromethyl)-2-(chloromethyl)-1,3,4-oxadiazole (1-2)

Bishydrazide 1-1 from Step A (43.2 g, 0.21 mol) in ACN (82 mL) was cooled to 5° C. Phosphorus oxychloride (32.2 g, 0.21 mol) was added, maintaining the temperature below 10° C. The mixture was heated to 80° C. and aged at this temperature for 24 h until HPLC showed less than 2 area % of 1-1. In a separate vessel, 260 mL of IPAc and 250 mL of water were mixed and cooled to 0° C. The reaction slurry was charged to the quench keeping the internal temperature below 10° C. After the addition, the mixture was agitated vigorously for 30 min, the temperature was increased to room temperature and the aqueous layer was cut. The organic layer was then washed with 215 mL of water, 215 mL of 5 wt % aqueous sodium bicarbonate and finally 215 mL of 20 wt % aqueous brine solution. HPLC assay yield after work up was 86-92%. Volatiles were removed by distillation at 75-80 mm Hg, 55° C. to afford an oil which could be used directly in Step C without further purification. Otherwise the product can be purified by distillation to afford 1-2.

¹H-NMR (400 MHz, CDCl₃): δ 4.8 (s, 2H) ppm.

¹³C-NMR (100 MHz, CDCl₃): δ 32.1, 115.8 (q, J=337 Hz), 156.2 (q, J=50 Hz), and 164.4 ppm.

Step C: Preparation of N-[(2Z)-piperazin-2-ylidene]trifluoroacetohydrazide (1-3)

To a solution of ethylenediamine (33.1 g, 0.55 mol) in methanol (150 mL) cooled at −20° C. was added distilled oxadiazole 1-2 from Step B (29.8 g, 0.16 mol) while keeping the internal temperature at −20° C. After the addition was complete, the resulting slurry was aged at −20° C. for 1 h. Ethanol (225 mL) was then charged and the slurry slowly warmed to −5° C. After 60 min at −5° C., the slurry was filtered and washed with ethanol (60 mL) at −5° C. Amidine 1-3 was obtained as a white solid (24.4 g, 99.5 area wt % pure by HPLC).

¹H-NMR (400 MHz, DMSO-d₆): δ 2.9 (t, 2H), 3.2 (t, 2H), 3.6 (s, 2H), and 8.3 (b, 1H) ppm. ¹³C-NMR (100 MHz, DMSO-d₆): δ 40.8, 42.0, 43.3, 119.3 (q, J=350 Hz), 154.2, and 156.2 (q, J=38 Hz) ppm.

Step D: Preparation of 3-(trifluoromethyl)-5,6,7,8-tetrahydro[1,2,4]triazolo[4,3-α]pyrazine hydrochloride (14) A suspension of amidine 1-3 (27.3 g, 0.13 mol) in 110 mL of methanol was warmed to 55° C. 37% Hydrochloric acid (11.2 mL, 0.14 mol) was added over 15 min at this temperature. During the addition, all solids dissolved resulting in a clear solution. The reaction was aged for 30 min. The solution was cooled down to 20° C. and aged at this temperature until a seed bed formed (10 min to 1 h). 300 mL of MTBE was charged at 20° C. over 1 h. The resulting slurry was cooled to 2° C., aged for 30 min and filtered. Solids were washed with 50 mL of ethanol:MTBE (1:3) and dried under vacuum at 45° C.

¹H-NMR (400 MHz, DMSO-d₆): δ 3.6 (t, 2H), 4.4 (t, 2H), 4.6 (s, 2H), and 10.6 (b, 2H) ppm; ¹³C-NMR (100 MHz, DMSO-d₆): δ 39.4, 39.6, 41.0, 118.6 (q, J=325 Hz), 142.9 (q, J=50 Hz), and 148.8 ppm.

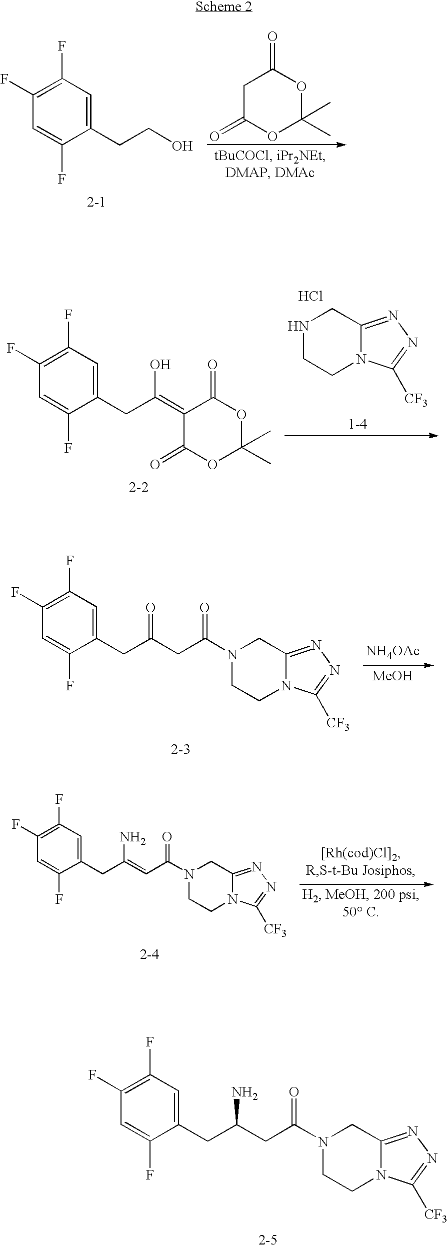

Step A: Preparation of 4-oxo-4-[3-(trifluoromethyl)-5,6-dihydro[1,2,4]triazolo[4,3-α]pyrazin-7(8H)-1-(2,4,5-trifluorophenyl)butan-2-one (2-3)

2,4,5-Trifluorophenylacetic acid (2-1) (150 g, 0.789 mol), Meldrum's acid (125 g, 0.868 mol), and 4-(dimethylamino)pyridine (DMAP) (7.7 g, 0063 mol) were charged into a 5 L three-neck flask N,N-Dimethylacetamide (DMAc) (525 mL) was added in one portion at room temperature to dissolve the solids. N,N-diisopropylethylamine (282 mL, 1.62 mol) was added in one portion at room temperature while maintaining the temperature below 40° C. Pivaloyl chloride (107 mL, 0.868 mol) was added dropwise over 1 to 2 h while maintaining the temperature between 0 and 5° C. The reaction mixture was aged at 5° C. for 1 h. Triazole hydrochloride 1-4 (180 g, 0.789 mol) was added in one portion at 40-50° C. The reaction solution was aged at 70° C. for several h. 5% Aqueous sodium hydrogencarbonate solution (625 mL) was then added dropwise at 20-45° C. The batch was seeded and aged at 20-30° C. for 1-2 h. Then an additional 525 mL of 5% aqueous sodium hydrogencarbonate solution was added dropwise over 2-3 h. After aging several h at room temperature, the slurry was cooled to 0-5° C. and aged 1 h before filtering the solid. The wet cake was displacement-washed with 20% aqueous DMAc (300 mL), followed by an additional two batches of 20% aqueous DMAc (400 mL), and finally water (400 mL). The cake was suction-dried at room temperature.

Step B: Preparation of (2Z)-4-oxo-4-[3-(trifluoromethyl)-5,6-dihydro[1,2,4]triazolo[4,3-α]pyrazin-7(8H)-yl]-1-(2,4,5-trifluorophenyl)but-2-en-2-amine (2-4)

A 5 L round-bottom flask was charged with methanol (100 mL), the ketoamide 2-3 (200 g), and ammonium acetate (110.4 g). Methanol (180 mL) and 28% aqueous ammonium hydroxide (58.6 mL) were then added keeping the temperature below 30° C. during the addition. Additional methanol (100 mL) was added to the reaction mixture. The mixture was heated at reflux temperature and aged for 2 h. The reaction was cooled to room temperature and then to about 5° C. in an ice-bath. After 30 min, the solid was filtered and dried to afford 2-4 as a solid; m.p. 271.2° C.

Step C: Preparation of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine(2-5)

Into a 500 ml flask were charged chloro(1,5-cyclooctadiene)rhodium(I) dimer {[Rh(cod)Cl]₂} (292 mg, 1.18 mmol) and (R,S) t-butyl Josiphos (708 mg, 1.3 mmol) under a nitrogen atmosphere. Degassed MeOH was then added (200 mL) and the mixture was stirred at room temperature for 1 h. Into a 4 L hydrogenator was charged the enamine amide 2-4 (118 g, 0.29 mol) along with MeOH (1 L). The slurry was degassed. The catalyst solution was then transferred to the hydrogenator under nitrogen. After degassing three times, the enamine amide was hydrogenated under 200 psi hydrogen gas at 50° C. for 13 h. Assay yield was determined by HPLC to be 93% and optical purity to be 94% ee.

The optical purity was further enhanced in the following manner. The methanol solution from the hydrogenation reaction (18 g in 180 mL MeOH) was concentrated and switched to methyl t-butyl ether (MTBE) (45 mL). Into this solution was added aqueous H₃PO₄ solution (0.5 M, 95 mL). After separation of the layers, 3N NaOH (35 mL) was added to the water layer, which was then extracted with MTBE (180 mL+100 mL). The MTBE solution was concentrated and solvent switched to hot toluene (180 mL, about 75° C.). The hot toluene solution was then allowed to cool to 0° C. slowly (5-10 h). The crystals were isolated by filtration (13 g, yield 72%, 98-99% ee); m.p. 114.1-115.7° C.

¹H NMR (300 MHz, CD₃CN): δ 7.26 (m), 7.08 (m), 4.90 (s), 4.89 (s), 4.14 (m), 3.95 (m), 3.40 (m), 2.68 (m), 2.49 (m), 1.40 (bs).

Compound 2-5 exists as amide bond rotamers. Unless indicated, the major and minor rotamers are grouped together since the carbon-13 signals are not well resolved:

¹³C NMR (CD₃CN): δ 171.8, 157.4 (ddd, J_{CF}=242.4, 9.2, 2.5 Hz), 152.2 (major), 151.8 (minor), 149.3 (ddd; J_{CF}=246.7, 14.2, 12.9 Hz), 147.4 (ddd, J_{CF}=241.2, 12.3, 3.7 Hz), 144.2 (q, J_{CF}=38.8 Hz), 124.6 (ddd, J_{CF}=18.5, 5.9, 4.0 Hz), 120.4 (dd, J_{CF}=19.1, 6.2 Hz), 119.8 (q, J_{CF}=268.9 Hz), 106.2 (dd, J_{CF}=29.5, 20.9 Hz), 50.1, 44.8, 44.3 (minor), 43.2 (minor), 42.4, 41.6 (minor), 41.4, 39.6, 38.5 (minor), 36.9.

The crystalline free base can also be isolated as follows:
(a) The reaction mixture upon completion of the hydrogenation step is charged with 25 wt % of Ecosorb C-941. The mixture is stirred under nitrogen for one h and then filtered. The cake is washed with 2 L/kg of methanol. Recovery of free base is about 95% and optical purity about 95% ee.
(b) The freebase solution in methanol is concentrated to 3.5-4.0 L/kg volume (based on free base charge) and then solvent-switched into isopropanol (IPA) to final volume of 3.0 L/kg IPA.
(c) The slurry is heated to 40° C. and aged 1 h at 40° C. and then cooled to 25° C. over 2 h.
(d) Heptane (7 L/kg) is charged over 7 h and the slurry stirred for 12 h at 22-25° C. The supernatant concentration before filtering is 10-12 mg/g.
(e) The slurry is filtered and the solid washed with 30% IPA/heptane (2 L/kg).
(f) The solid is dried in a vacuum oven at 40° C.
(g) The optical purity of the free base is about 99% ee.

### (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine dihydrogenphosphate monohydrate (MK-0431)

A 250 mL round bottom flask equipped with an overhead stirrer, heating mantle and thermocouple, was charged with 31.5 mL of isopropanol (IPA), 13.5 mL water, 15.0 g (36.9 mmol) of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine freebase and 4.25 g (36.9 mmol) of 85% aqueous phosphoric acid. The mixture was heated to 75° C. A thick white precipitate formed at lower temperatures but dissolved upon reaching 75° C. The solution was cooled to 68° C. and then held at that temperature for 2 h. A slurry bed of solids formed during this age time [the solution can be seeded with 0.5 to 5 wt % of small particle size (alpine milled) monohydrate]. The slurry was then cooled at a rate of 4° C./h to 21° C. and then held overnight. 105 mL of IPA was then added to the slurry. After 1 h the slurry was filtered and washed with 45 mL IPA (solids can also be washed with a water/IPA solution to avoid turnover to other crystal forms). The solids were dried on the frit with open to air. 18.6 g of solids were recovered. The solids were found to greater than 99.8% pure by HPLC area percentage (HPLC conditions same as those given above). The particle size distribution analysis of the isolated solids showed a mean PSD of 80 microns with 95% less than 180 microns. The crystal form of the solids was shown to be monohydrate by X-ray powder diffraction and thermogravimetric analysis.

### EXAMPLE 1

### In Vivo Study for Combination Therapy with a DPP-IV Inhibitor (Mk-0431) and a Cannabinoid CB₁ Receptor Antagonist/Inverse Agonist (Compound of Formula III) (Effect on Obesity/Food Intake and Glucose/Insulin)

DIO mice are treated simultaneously with an effective dose of Compound of Formula III and an effective dose of MK-0431.

### Materials and Methods:

Male C57BL/6J mice (CLEA Japan Inc., 12-16 months old at the beginning of the drug administration) are used. Mice are given water and regular pellet chow (CE-2, CLEA Japan Inc.) ad libitum. They are kept in an animal room which is maintained at 23±2° C. temperature, 55±15% relative humidity and on a 12-hr light-dark cycle (7:00-19:00) during a quarantine and acclimatization period of 1 week. Before the start of drug administration, mice are fed a MHF diet (Oriental BioService Co., Tokyo, Japan) for at least 2 months until the body weight gain reaches a plateau. After the body weight gain reaches a plateau, the diet is changed to a powder MHF diet. The powder MHF diet is given by powder feeder (small dishes). Diet and dishes are changed everyday, and daily food intake is measured. During this period, animals are orally administered vehicle (0.5% methylcellulose in distilled water) by gavage once-daily. After the stable feeding is observed, the amount of new food is adjusted to daily food intake+0.3 g, to minimize the amount of spilled food. After the acclimation period, the MHF diet-fed mice are divided into two groups to match average values of body weight and food intake (n=8-12). One of the groups is orally administered vehicle while the second group is administered a combination of Compound of Formula III and MK-0431. MK-0431 is given at a dose of 100 mg/kg once-daily and Compound of Formula III is given at a dose of 10 mg/kg once a day for 1.5 months by gavage, respectively. The administration is done one and half hours before the beginning of the dark period following the measurement of body weight. Food and body weight are measured. At the end of the treatment period, animals are fasted overnight and an oral glucose tolerance test is performed.

Effective combinations result in body weight loss of >5% and a statistically significant reduction in glucose and/or insulin, and/or improvement in an oral glucose tolerance test in the treated group compared to the vehicle treated group.

### EXAMPLE 2

### Human Study for Combination Therapy with a DPP-IV Inhibitor (Mk-0431) and a Cannabinoid CB₁ Receptor Antagonist/Inverse Agonist (Compound of Formula III) (Effect on Obesity/Food Intake and Glucose/Insulin)

### Materials and Methods:

A suitable number of people with a BMI≧30 who have impaired fasting plasma glucose levels, impaired glucose tolerance, or elevated serum insulin, indicative of a prediabetic insulin resistant state, or who may have elevated serum glucose levels, indicative of type II diabetes, are advised to diet and increase their physical activity. After a two-week placebo run-in period, which includes a standardized program of diet, physical activity, and lifestyle changes, the patients are randomized into 4 treatment groups: placebo; an effective dose of MK-0431, such as 100 mg; an effective dose of Compound of Formula III, such as 10 mg; and an effective dose of Compound of Formula III plus an effective dose of MK-0431. Compound of Formula III is given once or more per day, as previously determined to be effective. MK-0431 is given once or more per day, as previously determined to be effective. The two compounds may be given in a single dosage form. Patients are treated for 6 months, body weights are measured every two to four weeks, and appetite, hunger, satiety are measured every two to twelve weeks using standard questionnaires. Serum glucose and insulin levels are determined at day 0, at four to twelve week intervals, and after the final dose.

Effective combinations result in body weight loss of ≧5% and an improvement in serum insulin levels, indicative of improved insulin sensitivity and/or lower fasting blood glucose levels.

### EXAMPLE 3

### Non Diabetic Rodent Model of Metabolic Syndrome: Study for Combination Therapy with a DPP-IV Inhibitor (Mk-0431) and a Cannabinoid CB₁ Receptor Antagonist/Inverse Agonist (Compound of Formula III) Optionally Containing an Anti-Hypersensitive Agent and/or an Anti-Dyslipidemic Agent. (Effect Blood Pressure, Serum Insulin Levels, Triglyceride Levels, and Fatty Acid Levels)

The following experiment demonstrates the ability of the composition to lower blood pressure in an animal model of Metabolic Syndrome. This experiment uses a non-diabetic rodent model where blood insulin levels, blood pressure and serum triglycerides are elevated but serum glucose levels are within normal limits.

### Materials and Methods

Male, Sprague-Dawley rats Harlan Sprague Dawley, Indianapolis, Ind.), initially weighing 175-199 g are used for all experiments. Prior to dietary manipulation, all rats are fed Purina Rat Chow (no. 5012; St. Louis, Mo.) and water ad libitum and maintained on a 12-h (0600-1800 h) light-dark cycle. The rats are then placed on a diet (TD 78463; Harlan Teklad, Madison, Wis.) which provides 60% of total calories as fructose. The fructose-enriched diet is given for 11 days, during which time the rats are acclimated to the procedure of blood pressure measurement. Ambient temperature is kept at 30 C. The equipment used includes magnetic animal holders connected with manual scanner (model 65-12, IITC, Inc., Woodland Hills, Calif.), pulse amplifier (model 59, IITC, Inc.), and dual-channel recorder (model 1202, Linear Intrs. Corp., Reno, Nev.).

At the end of the initial dietary period, blood pressure is determined, and rats randomly divided into two groups. Both groups are maintained on the fructose-enriched diet, but one group is gavaged with a combination of MK-0431 (such as 100 mpk PO) and Compound of Formula III, optionally with an antihypertensive agent such as enalapril or losartan and/or an anti-lipid agent such as simvastatin, whereas the other group is treated in the same manner with vehicle alone. Blood pressure is measured once per week, before and after doses of either the combination or vehicle (8 weeks of treatment). In both instances, the general procedure is similar. Rats are removed from the animal room and taken to the laboratory at 0900 h. They are allowed free access to water and are kept in a quiet area before the blood pressure is measured at 1300 h. The tail-cuff method, without external preheating, is used to measure the systolic blood pressure. The systolic blood pressure is measured in the conscious state and has been shown with this technique to be similar to that obtained by direct arterial cannulation. The final blood pressure determinations were performed on the afternoon following the last morning dose of the combination or vehicle. In approximately half of the rats studied, tail vein blood is removed at 1300 h (four hours after removal of food), centrifuged, frozen, and later assayed for plasma glucose, insulin, and triglyceride concentrations. Plasma free fatty acid concentration is assayed enzymatically by the ACS-ACOD method using a commercial kit (Waro Chemicals Inc., Richmond, Va.).

The animal model used in this example has many of the features of Metabolic Syndrome. Fructose fed rats do not have increased blood glucose and therefore this is not a diabetic model. However, these rats do show increased serum insulin, increased triglycerides and free fatty acid concentration and increased blood pressure. Thus, this animal model is the animal model for Metabolic Syndrome.

Effective compositions improve the characteristic cluster of symptoms associated with Metabolic Syndrome. Effective compositions lower at least two of the symptoms of Metabolic Syndrome: blood pressure, blood insulin, free fatty acid, bodyweight and triglyceride levels in a non-diabetic rat model where blood glucose levels remain normal.

Additional animal models can be used, including BRS3 KO mice (Ohki-Hamazki et al, Nature 390: 165 (1997) and diet-induced obese and hypertensive dogs (Hall et al, Am. J. Hypertension. 14: 103S-115S (2001)).

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in responsiveness of the mammal being treated for any of the indications with the compounds of the invention indicated above. The specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A pharmaceutical composition comprising a cannabinoid CB₁ receptor antagonist/inverse agonist selected from the group consisting of:
N-[3-(4-chlorophenyl)-1-methyl-2-phenylpropyl]-2-(2-pyridyloxy)-2-methylpropanamide;
N-[3-(4-chlorophenyl)-2-(3,5-difluorophenyl)-1-methylpropyl]-2-(2-pyridyloxy)-2-methylpropanamide;
N-[3-(4-chlorophenyl)-1-methyl-2-phenyl-propyl]-2-(5-chloro-2-pyridyloxy)-2-methylpropanamide;
N-[3-(4-chlorophenyl)-2-(3-pyridyl)-1-methylpropyl]-2-(5-trifluoromethyl-2-pyridyloxy)-2-methylpropanamide;
N-[3-(4-chlorophenyl)-2-(3-cyanophenyl)-1-methylpropyl]-2-(5-trifluoromethyl-2-pyridyloxy)-2-methylpropanamide;
N-[3-(4-chlorophenyl)-2-(5-chloro-3-pyridyl)-1-methylpropyl]-2-(5-trifluoromethyl-2-pyridyloxy)-2-methylpropanamide;
N-[3-(4-chlorophenyl)-2-(5-methyl-3-pyridyl)-1-methylpropyl]-2-(5-trifluoromethyl-2-pyridyloxy)-2-methylpropanamide;
N-[3-(4-chlorophenyl)-2-(5-cyano-3-pyridyl)-1-methylpropyl]-2-(5-trifluoromethyl-2-pyridyloxy)-2-methylpropanamide;
N-[3-(4-chlorophenyl)-2-(3-methylphenyl)-1-methylpropyl]-2-(5-trifluoromethyl-2-pyridyloxy)-2-methylpropanamide;
N-[3-(4-chlorophenyl)-2-phenyl-1-methylpropyl]-2-(4-trifluoromethyl-2-pyridyloxy)-2-methylpropanamide;
N-[3-(4-chlorophenyl)-2-phenyl-1-methylpropyl]-2-(4-trifluoromethyl-2-pyrimidyloxy)-2-methylpropanamide;
N-[3-(4-chlorophenyl)-1-methyl-2-(thiophen-3-yl)propyl]-2-(5-chloro-2-pyridyloxy)-2-methylpropanamide;
N-[3-(5-chloro-2-pyridyl)-2-phenyl-1-methylpropyl]-2-(5-trifluoromethyl-2-pyridyloxy)-2-methylpropanamide;
N-[3-(4-fluorophenyl)-2-(3-cyanophenyl)-1-methylpropyl]-2-(5-trifluoromethyl-2-pyridyloxy)-2-methylpropanamide;
N-[3-(4-methoxy-phenyl)-2-(3-cyanophenyl)-1-methylpropyl]-2-(5-trifluoromethyl-2-pyridyloxy)-2-methylpropanamide;
N-[3-(4-chlorophenyl)-2-(3-cyanophenyl)-1-methylpropyl]-2-(6-trifluoromethyl-4-pyrimidyloxy)-2-methylpropanamide;
N-[2-(3-cyanophenyl)-1,4-dimethylpentyl]-2-(5-trifluoromethyl-2-pyridyloxy)-2-methylpropanamide;
N-[2-(3-cyanophenyl)-3-cyclobutyl-1-methylpropyl]-2-(5-trifluoromethyl-2-pyridyloxy)-2-methylpropanamide;
N-[2-(3-cyanophenyl)-1-methyl-heptyl]-2-(5-trifluoromethyl-2-pyridyloxy)-2-methylpropanamide;
N-[2-(3-cyanophenyl)-3-cyclopentyl-1-methylpropyl]-2-(5-trifluoromethyl-2-pyridyloxy)-2-methylpropanamide;
N-[2-(3-cyanophenyl)-3-cyclohexyl-1-methylpropyl]-2-(5-trifluoromethyl-2-pyridyloxy)-2-methylpropanamide;
3-{1-[bis(4-chlorophenyl)methyl]azetidin-3-ylidene}-3-(3,5-difluorophenyl)-2,2-dimethylpropanenitrile;
1-{1-[1-(4-chlorophenyl)pentyl]azetidin-3-yl}-1-(3,5-difluorophenyl)-2-methylpropan-2-ol;
3-((S)-(4-chlorophenyl){3-[(1S)-1-(3,5-difluorophenyl)-2-hydroxy-2-methylpropyl]azetidin-1-yl}methyl)benzonitrile;
3-((S)-(4-chlorophenyl){3-[(1S)-1-(3,5-difluorophenyl)-2-fluoro-2-methylpropyl]azetidin-1-yl}methyl)benzonitrile;
3-((4-chlorophenyl){3-[1-(3,5-difluorophenyl)-2,2-dimethylpropyl]azetidin-1-yl}methyl)benzonitrile;
3-((1S)-1-{1-[(S)-(3-cyanophenyl)(4-cyanophenyl)methyl]azetidin-3-yl}-2-fluoro-2-methylpropyl)-5-fluorobenzonitrile;
3-[(S)-(4-chlorophenyl)(3-{(1S)-2-fluoro-1-[3-fluoro-5-(4H-1,2,4-triazol-4-yl)phenyl]-2-methylpropyl}azetidin-1-yl)methyl]benzonitrile; and
5-((4-chlorophenyl){3-[(1S)-1-(3,5-difluorophenyl)-2-fluoro-2-methylpropyl]azetidin-1-yl}methyl)thiophene-3-carbonitrile;
or a stereoisomer, pharmaceutically acceptable salt, hydrate, and crystalline form thereof;
and a dipeptidyl peptidase-IV inhibitor which is (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine of the structural formula: 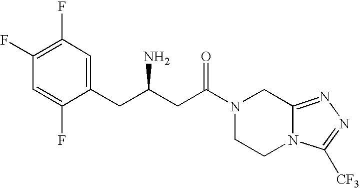
or a pharmaceutically acceptable salt, hydrate, and crystalline form thereof.

2. The pharmaceutical composition of claim 1 wherein said pharmaceutically acceptable salt of said dipeptidyl peptidase-IV inhibitor is the dihydrogenphosphate salt of the structural formula: 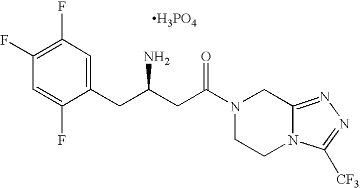
or a pharmaceutically acceptable hydrate thereof.

3. The pharmaceutical composition of claim 2 wherein said CB₁ receptor antagonist/inverse agonist is N-[(1S,2S)-3-(4-chlorophenyl)-2-(3-cyanophenyl)-1-methylpropyl]-2-methyl-{[5-(trifluoromethyl)pyridin-2-yl]oxy}propanamide of the formula: 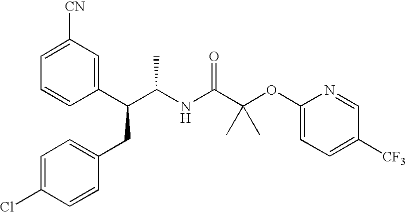
or a pharmaceutically acceptable salt, hydrate, or crystalline form thereof.

4. The pharmaceutical composition of claim 3 wherein said dihydrogenphosphate salt is in the form of a crystalline monohydrate.

5. A method of treating a condition selected from diabetes, a diabetes-related disorder, obesity, and an obesity-related disorder comprising administering to a subject in need thereof a therapeutically effective amount of the composition of claim 3.

6. The method of claim 5 wherein said dihydrogenphosphate salt is in the form of a crystalline monohydrate.

7. The method of claim 5 wherein said condition is diabetes.

8. The method of claim 5 wherein said condition is obesity.

9. The method of claim 5 wherein said diabetes-related disorder is selected from the group consisting of hyperglycemia, prediabetes, impaired glucose tolerance, impaired fasting glucose, obesity, dyslipidemia, hyperlipidemia, hypertriglyceridemia, hypercholesterolemia, low HDL levels, high LDL levels, atherosclerosis, hypertension, sleep apnea, polycystic ovarian syndrome, and Metabolic Syndrome.

10-11. (canceled)

12. The method of claim 5 wherein said cannabinoid CB₁ receptor antagonist/inverse agonist and said dipeptidyl peptidase-IV inhibitor are administered together in a single pharmaceutical composition in association with a pharmaceutically acceptable carrier or diluent.
